(19) United States
(12) Patent Application Publication
(10) Pub. No.: US 2014/0045809 A1
(43) Pub. Date: Feb. 13, 2014

(54) CORROLES FOR NEUROPROTECTION AND NEURORESCUE

(71) Applicant: TECHNION RESEARCH AND DEVELOPMENT FOUNDATION LTD., Haifa (IL)

(72) Inventors: Moussa B.H. YOUDIM, Haifa (IL); Silvia MANDEL, Haifa (IL); Tamar AMIT, Haifa (IL); Orit BAR-AM, Kiryat Tivon (IL); Lana KUPERSHMIDT, Kiryat Bialik (IL); Zeev GROSS, Petach Tikva (IL); Atif MAHAMMED, Muawya Village (IL); Irena SALTSMAN, Haifa (IL); Zoya OKUN, Nesher (IL)

(73) Assignee: TECHNION RESEARCH AND DEVELOPMENT FOUNDATION LTD., Haifa (IL)

(21) Appl. No.: 14/016,262

(22) Filed: Sep. 3, 2013

Related U.S. Application Data
(62) Division of application No. 12/865,793, filed on Dec. 21, 2010, now abandoned.
(60) Provisional application No. 61/025,043, filed on Jan. 31, 2008.

(51) Int. Cl. A61K 31/555

(52) U.S. Cl. CPC A61K 31/555
USPC 514/184

(57) ABSTRACT

Transition metal complexes of amphiphilic corroles, optical isomers and pharmaceutically acceptable salts thereof are useful for neuroprotection and neurorescue, particularly for treatment of diabetes and neurodegenerative diseases. The amphiphilic corrole is preferably a 5,10,15-tris-aryl- or 5,10,15-tris-CF₃-corrole, and said transition metal complex more preferably has the formula I defined in the specification. Also provided are propargyl-containing corroles carrying one or more radicals substituted by a propargylamino group or one or more nitrogen-containing heteroaryl radicals substituted by propargyl at the ring N atom.

### RELATED APPLICATIONS

The present application is a divisional application which claims benefit under 35 U.S.C. §120 to U.S. patent application Ser. No. 12/865, 793, which is a US National Phase of PCT Application No. PCT/IL2009/000121, filed Feb. 1, 2009, which claims the benefit under 35 U.S.C. §119(e) of U.S. Provisional Application 61/025,043 filed Jan. 31, 2008, the disclosures of which are incorporated herein by reference.

### FIELD OF THE INVENTION

The present invention relates to neuroprotection and neurorescue and, particularly, to the use of transition metal complexes of corroles as neuroprotective and neurorescuing agents for treatment of diabetes and neurodegenerative diseases and disorders, and to some new metal complexes of corroles.

### BACKGROUND OF THE INVENTION

Research into the causes of neurodegenerative disorders such as Parkinson's disease (PD), Alzheimer's disease (AD), Huntington disease (HD) and amyotrophic lateral sclerosis (ALS, a motor neuron disease), as well as of diabetes, have been fueled in part by frustration over the shortcomings of the drugs available for treatment. The true aim of therapy must ultimately be to identify the disease process before symptoms are recognized and to develop new drugs to prevent the progress of cell death. Novel therapeutic strategies for neurodegenerative diseases are clearly needed that should focus on either neuroprotection or neurorestoration as to slow down the death process or cause neurorestoration via neurogenesis, respectively. Neuropathological studies converge into a common concept, viewing neurodegenerative diseases as multifactorial, whereby several mechanisms are implicated in a cascade of events involving many biochemical and signaling pathways.

Ongoing research has clearly indicated that therapies aimed at blocking oxidative processes involving reactive oxygen species (ROS), nitric oxide (NO) production, diet supplied antioxidant polyphenols, antiapototic drugs, such as calcium channel and caspases inhibitors, bioenergetic drugs inhibitors of glutamate transmission and iron chelators would be a prominent approach in monotherapy or as part of antioxidant cocktail formulation for the treatment of these diseases (Youdim et al., 2005; Mandel et al., 2007a). The rationale is that the simultaneous manipulation of multiple desired targets in the central nervous system (CNS) will exert higher therapeutic effectiveness (Mandel et al., 2007b). Iron accumulation and deposition in the brain can cause a vast range of disorders of the CNS including PD, AD, Lewy body disease, HD, ALS, multiple sclerosis (MS), aceruloplasminemia, Friedreich's ataxia, and neurodegeneration with brain iron accumulation. Furthermore, iron is known to accumulate in the brain as a function of age, in a cell-specific manner, particularly in brain regions that are susceptible to neuron damage. It is well established that iron participates in the Fenton chemistry, reacting with hydrogen peroxide (H₂O₂) to produce the most reactive of all ROS, the hydroxyl radical. The formation of the latter, combined with the depletion of endogenous antioxidants, particularly tissue reduced glutathione (GSH), the most common pathway of iron deposition in the brain, leads to oxidative stress (OS). Iron also facilitates the decomposition of lipid peroxides to produce highly cytotoxic oxygen-related free radicals. Free radicals-related OS causes damage to DNA, lipids, proteins and ultimately cell death associated with neurodegenerative diseases.

It is widely accepted that ROS participate in the development and progression of Diabetes Mellitus (DM) and its complications. Beta cells have a reduced capacity to scavenge free radicals and are very sensitive to ROS and reactive nitrogen species (RNS) action because of their poor antioxidant system (low levels of GSH and of the H₂O₂ decomposing enzyme catalase). These facts are believed to be responsible for the high sensitivity of insulin producing cells to various insults leading to their destruction and resulting in diabetes.

Intriguingly, several lines of evidence indicate that the developmental causes of PD, AD and DM share common etiologies, particularly regarding their common neuroendocrine origin and the role of oxidative/nitrosative stress in the process of cell death. Thus, it can be speculated that neurodegenerative diseases and DM, can be prevented, and/or the damaging effects of ROS reduced, by administration of free radical scavengers and antioxidant compounds, as part of a polypharmacology approach or as a multimodal acting cytoprotective drug intended to treat the several etiologies of these diseases.

In addition to ROS, prominent role of RNS in the early stages of numerous diseases is indicated, even though the final outcome in terms of the specific malfunction or illness differs substantially in each case (Shah et al., 2007). Accordingly, endogenous and exogenous (diet-derived) antioxidants are expected to play a major role in preventing oxidative/nitrosative damage to vital biomolecules, but many compounds that display potent anti-oxidant properties in vitro fail in displaying beneficial in vivo effects. One clue for a possible resolution of this apparent conflict comes from increasing evidence indicating a crucial role for nitrated protein residues (mainly nitrotyrosine) in all these diseases (Mohiuddin et al., 2006). This suggests that excessive RNS are not neutralized by the natural anti-oxidants as efficiently as the ROS.

One particularly detrimental species is peroxynitrite (HOONO) (PN), formed via the ultra-fast interaction between superoxide anion (O₂⁻) and NO, whose homolytic cleavage leads to hydroxyl radical (or CO₃⁻ in CO₂-rich environment) and .NO₂, the most reactive ROS and RNS (Goldstein et al., 2005). These species (and secondary radicals derived from them) are considered to be involved in the damage of a very large variety of molecules (Szabo et al., 2007). In contrast to other ROS and RNS precursors, peroxynitrite (PN) is particularly toxic, since there is no intrinsic biological defense system against it; and all natural catalytic anti-oxidants (including SOD and catalase enzymes) do not react with PN faster than the vital biomolecules (Olmos et al., 2007). This calls for the development of synthetic reagents that could act on and neutralize PN by one or more of the following ways: a) interfering with its formation by eliminating its precursors (O₂⁻ and NO); b) rapidly decomposing it to biologically benign products; c) repairing the damage caused by it. The major advances in this field are with metalloporphyrin-based decomposition catalysts of peroxynitrite, which appear to be efficient for treating certain diseases that are related to oxidative/nitrosative stress (Wu et al., 2003; Liang et al., 2007).

A note of caution must, however, be taken when translating in vitro findings into any particular in vivo system (Schlieve et al, 2006). For example, the prominent anti-cancer drug cisplatin becomes therapeutically inactive very fast because of its strong binding to serum albumin. This kind of limitation is even more severe for treating neurodegenerative diseases because the potential drug must be able to cross the blood-brain-barrier (BBB).

Corroles are tetrapyrrole macrocycles closely related to porphyrins, with one carbon atom less in the outer periphery and one NH proton more in their inner core. The corroles are much less known than porphyrins and their synthesis was considered to be very complex, until a simple procedure for corrole synthesis has been disclosed in U.S. Pat. No. 6,541,628.

U.S. Pat. No. 6,730,666 discloses porphyrins and corroles useful for inhibition of cell proliferation mediated by growth factor receptor tyrosine kinase activity, for example, for inhibition of angiogenesis, or vascular smooth muscle cell proliferation in disorders including atherosclerosis, hyperthrophic heart failure and postsurfical restenosis, and of cell proliferation and migration in the treatment of primary tumors and metastasis. The corroles defined in this patent are metal-free and positively charged and the sole corrole disclosed therein, 5,10,15-tris[2,3,5,6-tetrafluorophenyl-4-(N-methyl-2-pyridylium)]-21H,23H-corrole triiodide, was shown to inhibit the appearance of lung metastasis in an animal model.

Corroles can chelate a variety of metal ions and, once bound, the mettalocorroles are much less prone to dissociation of potentially toxic metal than analogous metalloporphyrins. Co-inventors in the present application have demonstrated in two recent publications demonstrated that iron(III) and manganese(III) complexes of 5,10,15-tris(pentafluorophenyl)-2,17-bis(sulfonic acid)-corrole (disclosed in U.S. Pat. No. 6,939,963) are excellent catalysts for decomposition of two important reactive molecules, hydrogen peroxide and peroxynitrite (Mahammed et al., 2005; Mahammed and Gross, 2006, Aviv et al., 2007). Firm evidence in favor of a disproportionation mechanism were provided for both H₂O₂ and peroxynitrite (HOONO): they first serve as oxidants for transferring the manganese(III) corrole into the (oxo)manganese(V) complex, which then utilizes the same molecules as reductants for returning to manganese(III). Less detailed mechanistic insight was obtained for the iron complex, but its catalytic rates were found to be faster than those of the Mn complex and it apparently induced isomerization rather than disproportionation of peroxynitrite. The very fast action of the iron complex and the unique mechanism adopted by the manganese corrole (no other manganese complexes are capable of decomposing ROS and RNS catalytically in the absence of co-reductants, Gershman et al., 2007) suggest a significant added value of these complexes in the continuous efforts devoted to the development of synthetic catalysts that may either neutralize or avoid the formation of reactive oxygen and nitrogen species.

### SUMMARY OF THE INVENTION

In one aspect, the present invention relates to the use of a transition metal complex of an amphiphilic corrole, an optical isomer or a pharmaceutically acceptable salt thereof, for neuroprotection and neurorescue, particularly for the treatment of diabetes or of a neurodegenerative disease, disorder or condition.

The amphiphilic corrole is preferably a 5,10,15-tris-aryl- or 5,10,15-tris-CF₃-corrole, and said transition metal complex of the amphiphilic corrole more preferably has the formula I as defined hereinafter in the specification.

The present invention further provides a propargyl-containing corrole carrying one or more radicals substituted by a propargylamino group or one or more nitrogen-containing heteroaryl radicals substituted by propargyl at the ring N atom, and pharmaceutical compositions comprising them.

The propargyl-containing corrole is preferably a transition metal complex of an amphiphilic corrole of the formula I as defined hereinafter in the specification.

### BRIEF DESCRIPTION OF THE FIGURES

The corrole metal complexes are presented in the legends of the figures below by a short abbreviation in bold as used herein in the specification. The full names of the compounds are shown in the beginning of the section Examples and their structural formulas are depicted in Schemes 1 and 2, at the end of the description, just before the References.

FIG. 1 shows decomposition of 385 μm peroxynitrite at pH 7.4 and 25° C. monitored at λ=302 nm as a function of the concentration of manganese(III) corrole 4C-Mn. Inset: Plot used for the determination of the catalytic rate constant.

FIGS. 2A-2B show in-vivo fluorescence-based imaging of a live mouse before (FIG. 2A) and 30 minutes after (FIG. 2B) intraperitoneal injection of 19 mg/kg of the gallium corrole complex 1C-Ga. The results are indicative of the crossing of the blood brain barrier (BBB) by 1C-Ga, the fluorescent analog of 1C-Fe and 1C-Mn.

FIGS. 3A-3C show protective effect of corrole 1C-Fe, on clonal beta cell lines against hydrogen peroxide toxicity. (3A) protective effect of corrole 1C-Fe on INS-1E cells treated with increasing hydrogen peroxide (H₂O₂) concentrations. (3B) dose-dependent protective effect of corrole 1C-Fe on RIN-m cells treated with H₂O₂ (35 μM). (3C) corrole 1C-Fe shows no toxicity when applied alone (without H₂O₂) at concentrations up to 50 μM. The results represent the mean±SEM of three independent experiments performed under the same conditions. *p<0.05 versus vehicle-treated cells.

FIG. 4 displays protective effect of iron(III) corroles 1C-Fe and 5C-Fe (in concentrations of 1 and 20 μM) relative to their structurally related porphyrin 1P-Fe, against toxicity induced by H₂O₂ (35 μM) in RIN-m cells. Cell injury was evaluated by the MTT test. The results represent the mean±SEM of a representative experiment that was repeated twice with similar results performed under the same conditions. *p<0.001 vs vehicle-treated cells, #p<0.01 vs corresponding porphyrin.

FIGS. 5A-5B show cytoprotective effect of corroles and porphyrins on RIN-m cell line exposed to H₂O₂. (5A) protective effect of iron(III) corrole 1C-Fe and structurally related porphyrin 1P-Fe. (5B) inefficiency of manganese(III) complexes 1C-Mn, 2C-Mn and structurally related porphyrin 2P-Mn to protect against H₂O₂ induced damage. All concentrations are in μM. *p<0.004, #p<0.008.

FIGS. 6A-6B show the protective effect of iron(III) corroles 1C-Fe and 5C-Fe against toxicity induced by the nitric oxide donor linsidomine (SIN-1), in INS-1E cells and RIN-m cells, respectively. The results represent the mean±SEM of three independent experiments performed under the same conditions. *p<0.05 versus vehicle-treated cells.

FIGS. 7A-7C display comparative effects between (7A) the positively charged manganese(III) corrole 2C-Mn and its negatively charged analog 1C-Mn; (7B) the manganese(III) positively-charged corrole 2C-Mn and the structurally related porphyrin 2P-Mn; (7C) the negatively-charged iron(III) corrole 1C-Fe and the structurally related porphyrin 1P-Fe in RIN-m cells exposed to SIN-1. The results are the mean±standard error; experiments were repeated twice with similar results performed under the same conditions. *p<0.005, ^{#}p<0.01

FIGS. 8A-8B display neuroprotective and neurorescue effects of iron(III) corroles 1C-Fe and 5C-Fe against hydrogen peroxide-induced cell death in human neuroblastoma SH-SY5Y cell line. (8A) dose dependent neuroprotective effect of corroles 1C-Fe and 5C-Fe on SH-SY5Y cells. (8B) neurorescue of SH-SY5Y cells with corroles 1C-Fe and 5C-Fe, added 0.5, 1.5 and 3 hours after insult with H₂O₂. *p<0.01 vs. untreated cells.

FIGS. 9A-9D show comparative effects of iron(III) corrole 1C-Fe versus structurally related porphyrin 1P-Fe in SH-SY5Y cells exposed to H₂O₂. compounds 1C-Fe and 1P-Fe (1-50 μM) were added 30 min before insult with H₂O₂ (200 μM) for a subsequent 24 h period. (9A) cell viability was evaluated by MTT test, and (9B) cell death was assessed using the apoptotic cell death detection ELISA kit. (9C) the drug (1C-Fe) shows no toxicity when given alone (without H₂O₂) at concentrations up to 50 μM. The graphs present results expressed as percentage of untreated control. Data are expressed as mean±SEM (n=6) of a representative experiment that was repeated twice with similar results.$p<0.001 vs control; *p<0.001 vs vehicle-treated cells, # p<0.001 vs corresponding porphyrin. (9D) Neurorescue effects of corrole 1C-Fe against H₂O₂-induced cell death in human neuroblastoma SH-SY5Y cell line. SH-SY5Y cells were plated in microtiter plates (96 wells) in DMEM-Eagle/F-12(HAM) (1:1), containing 10% FCS. SH-SY5Y cells were first exposed to 20 mM H₂O₂, followed by the addition of corrole 1C-Fe after 0.5-1.5 h and incubated for further 24 h. *p<0.005 vs untreated cells.

FIG. 10 shows comparative effect of iron(III) corrole 1C-Fe and porphyrin 1P-Fe complexes in SH-SY5Y cells exposed to different H₂O₂ concentrations.

FIGS. 11A-11B display neuroprotective and neurorescue effects of corroles 1C-Fe and 5C-Fe against SIN-1-induced cell death in SH-SY5Y cell line. (11A) a dose dependent neuroprotective effect of corroles 1C-Fe and 5C-Fe on SH-SY5Y cells. (11B) neurorescue of SH-SY5Y cells with corroles 1C-Fe and 5C-Fe added at the indicated time after the insult with SIN-1. *p<0.01 vs. untreated cells.

FIGS. 12A-12C show comparative effects of corroles 1C-Fe, 2C-Mn and 3C-Mn, and porphyrins 1P-Fe and 2P-Mn, against SIN-1-induced toxicity in SH-SY5Y cells. SH-SY5Y cells were pretreated with or without one of the complexes 30 min before exposure to SIN-1 and incubated for a subsequent 24 h period. (12A) cell viability was evaluated by MTT test; and (12B) cell death was assessed using the apoptotic cell death detection ELISA kit. The graphs present results expressed as percentage of untreated control. Data are expressed as mean±SEM (n=6) of a representative experiment that was repeated twice with similar results. $p<0.001 vs control; *p<0.001 vs. vehicle-treated cells; # p<0.001 vs. corresponding porphyrin. (12C) cytoprotection by iron(III) corrole 1C-Fe and manganese(III) corrole 3C-Mn in SH-SY5Y cells exposure to different SIN-1 concentrations. *p<0.005, #p<0.005

FIGS. 13A-13B show effects of corroles 1C-Fe, 2C-Mn and 3C-Mn on nitrotyrosine level in SIN-1-induced neurotoxicity model. SH-SY5Y cells were pretreated with or without the corroles (20 μM) for 30 min before exposure to SIN-1 (700 μM) and analyzed after a subsequent 24 h period. Nitrotyrosine was detected by fluorescence microscopy using a specific primary antibody. (13A) The images show representative fields obtained by the combination of DAPI nuclear staining (reflected in blue color) and nitrotyrosine-antibody (reflected in red color). (13B) graph that represents absolute values of six to eight separate fields expressed as mean±SEM. #p<0.001 vs control; *p<0.001 vs vehicle-treated cells.

FIGS. 14A-14B display neuroprotective and neurorescue effects of iron(III) corroles 1C-Fe and 5C-Fe against 6-hydroxydopamine (OHDA)-induced cell death in SH-SY5Y cell line. (14A) dose-dependent neuroprotective effect of corroles 1C-Fe and 5C-Fe on SH-SY5Y cells. (14B) neurorescue of SH-SY5Y cells with corroles 1C-Fe and 5C-Fe added 0.5, 1.5, 3 and 6 h after the insult with 6-OHDA. *p<0.01 vs. untreated cells.

FIGS. 15A-15C show comparative effects of corroles 1C-Fe, 2C-Mn and 3C-Mn and porphyrins 1P-Fe, 2P-Mn against 6-OHDA-induced toxicity. SH-SY5Y cells were pretreated with or without the complexes (20 μM) 30 min before exposure to 6-OHDA (40 μM) for a subsequent 24 h period. (15A) cell viability was evaluated by MTT test and (15B) cell death was assessed using the apoptotic cell death detection ELISA kit. (15C) for neurorescue, SH-SY5Y cells were first exposed to 40 μM 6-OHDA for 0.5-3 h, followed by the addition of the corroles 1C-Fe, 2C-Mn and 3C-Mn (20 μM) and incubated for further 24 h. The graphs present results expressed as percentage of untreated control. Data are expressed as mean±SEM (n=6) of a representative experiment that was repeated twice with similar results. $p<0.001 vs control;*p<0.001 vs vehicle-treated cells; # p<0.001 vs corresponding porphyrin.

FIGS. 16 A-N show effects of corroles 1C-Fe (E,F), 2C-Mn (I,J), and 3C-Mn (K,L), and porphyrins 1P-Fe (G,H) and 2P-Mn (M,N) against 6-OHDA-induced toxicity. SH-SY5Y cells were pretreated with or without the corroles (20 μM) 30 min before exposure to 6-OHDA (40 μM) for a subsequent 24 h period. Growth-associated protein (GAP-43) was detected by fluorescence microscopy using a specific primary antibody (FIGS. 16A,C,E,G,I,K and M for control, 40 μM 6-OHDA, 1C-Fe, 1P-Fe, 2C-Mn, 3C-Mn and 2P-Mn, respectively). Apoptotic nuclei were determined by terminal deoxynucleotidyl transferase-mediated UTP-digoxigenin nick end labeling analysis and DAPI-staining was applied for nuclear labeling (FIGS. 16B,D,F,H,J,L and N for control, 40 μM 6-OHDA, 1C-Fe, 1P-Fe, 2C-Mn, 3C-Mn and 2P-Mn, respectively). The images are representative fields from two independent experiments.

FIGS. 17A-17F show effects of corroles 1C-Fe, 2C-Mn, and 3C-Mn, on cleaved caspase-3 level in 6-OHDA-induced neurotoxicity model. SH-SY5Y cells were pretreated with or without the corroles (20 μM) 30 min before exposure to 6-OHDA (40 μM) for a subsequent 24 h period. 6-OHDA was detected by fluorescence microscopy using a specific primary antibody. (17A-E) the images are representative fields for control, 6-OHDA (40 μM), 1C-Fe, 2C-Mn and 3C-Mn, respectively. (17F) the graph represents absolute values of six to eight separate fields expressed as mean±SEM. #p<0.001 vs. control; *p<0.005 vs. vehicle—treated cells.

FIGS. 18A-18B compare protective effects of corrole 1C-Fe and porphyrin 1P-Fe against hydrogen peroxide induced toxicity in NSC-34 cell line. Compounds 1C-Fe and 1P-Fe (20 μM) were added 30 min before insult with the hydrogen peroxide (300 μM) for a subsequent 24 h period. (18A) Cell viability was evaluated by MTT test and (18B) cell death was assessed using the apoptotic cell death detection ELISA kit. The graphs present results expressed as percentage of untreated control. Data are expressed as mean±SEM (n=6) of a representative experiment that was repeated twice with similar results. $p<0.001 vs. control; *p<0.001 vs. vehicle-treated cells; # p<0.001 vs. corresponding porphyrin.

FIGS. 19A-19B display comparative effects of corroles 1C-Fe and 2C-Mn and porphyrins 1P-Fe and 2P-Mn against SIN-1 induced toxicity in NSC-34 cells. NSC-34 cells were pretreated with or without the corroles 1C-Fe and 2C-Mn and porphyrins 1P-Fe and 2P-Mn, respectively, 30 min before exposure to SIN-1 for a subsequent 24 h period. (19A) Cell viability was evaluated by MTT test; and (19B) cell death was assessed using the apoptotic cell death detection ELISA kit. The graphs present results expressed as percentage of untreated control. Data are expressed as mean±SEM (n=6) of a representative experiment that was repeated twice with similar results. $p<0.001 vs control;*p<0.001 vs. vehicle-treated cells; # p<0.001 vs. corresponding porphyrin.

FIGS. 20A-20B display neuroprotective effect of corroles 1C-Fe and 5C-Fe in a cell culture model of familial ALS. (20A) dose-dependent neuroprotective effect of corroles 1C-Fe and 5C-Fe on mouse motoneuronal NSC-34 G93A cells expressing mutant G93A-SOD1. (20B) neuroprotective effect of corroles 1C-Fe and 5C-Fe on SH-SY5Y G93A cells expressing mutant G93A-SOD1. #p<0.001 vs cells that do not express the mutation (control).

FIGS. 21A-D shows cellular uptake of the fluorescent anionic corrole complex 1C-Ga (20 μM) by RIN-m cell line. (21A) nuclei staining by DAPI, (21B) corrole detection by fluorescence, (21C) merged images of FIGS. 21A and 21B, (21D) same as 21B, but in blank and white.

FIG. 22 shows cellular uptake of the fluorescent anionic corrole complex 1C-Ga (20 μM) by SH-SY5Y cell line. (21A) nuclei staining by DAPI, (21B) corrole detection by fluorescence, (21C) merged images of FIGS. 21A and 21B, (21D) same as 21B, but in blank and white.

### DETAILED DESCRIPTION OF THE INVENTION

The present invention relates to compositions and methods concerning the use of a metalated corrole, an optical isomer or a pharmaceutically acceptable salt thereof as a neuroprotective and neurorescuing agent.

The metalated corrole for use in the invention is preferably an iron- or manganese 5,10,15-tris-aryl or 5,10,15-tris-CF₃ corrole.

In one preferred embodiment, the corrole has the formula I:

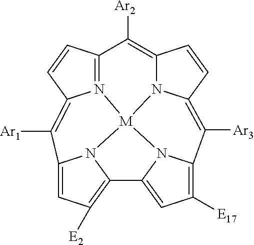

wherein:

Ar₁, Ar₂ and Ar₃, the same or different, each is selected from CF₃ or a carboaryl, heteroaryl or mixed carboaryl-heteroaryl radical;

M is a metal selected from Mn, Fe, Ru, Co, V, Cr, or Cu; and

E₂ and E₁₇, the same or different, each is H, SO₃H, SO₂N—R₁R₂, CO₂H, CO₂R or CON—R₁R₂; R is C₁-C₈ alkyl or C₆-C₁₂ aryl; and R₁ and R₂, the same or different, each is H, C₁-C₈ alkyl optionally substituted by —COOH, C₂-C₈ alkynyl, C₆-C₁₂ aryl or together with the N atom to which they are attached form a saturated 5-6 membered ring optionally containing a further heteroatom selected from O, S and N.

As defined herein, the term “carboaryl”, by itself or as part of the mixed carboaryl-heteroaryl radical, refers to a monocyclic or bicyclic aromatic radical having from 6 to 12 carbon atoms, such as phenyl, biphenyl or naphthyl optionally mono- or poly-substituted by one or more halogen atoms, or by radicals including, but not limited to, C₁-C₈ alkyl, C₁-C₈ alkoxy, nitro, hydroxyl, SO₃H, —NR₁R₂, —N⁺R₁R₂R₃, or —N—R₁—NH₂, wherein R₁, R₂ and R₃, the same or different, each is H, C₁-C₈ alkyl, C₂-C₈ alkynyl, C₆-C₁₂ aryl, C₆-C₁₂aryl-C₁-C₈ alkyl or R₁ and R₂ together with the N atom to which they are attached form a saturated 5-6 membered ring optionally containing a further heteroatom selected from O, S and N. The term “aryl” herein refers to the same definitions as the carboaryl.

As used herein, the term “alkyl” alone or as part of a radical such as “aralkyl” refers to a straight or branched C₁-C₈ alkyl radical such as, but not limited to, methyl, ethyl, n-propyl, isopropyl, n-butyl, isobutyl, tert-butyl, n-heptyl, 2,2-dimethylpropyl, pentyl, n-hexyl, n-heptyl and octyl, and preferably has 1-4 carbon atoms, more preferably methyl, ethyl, and propyl. The term “alkynyl” refers to a straight or branched C₂-C₈ alkynyl radical such as, but not limited to, ethynyl, propargyl, butynyl, and the like, in which the triple bond is in the n-position, and is preferably propargyl. The term “halogen” as used herein refers to fluoro, chloro, bromo or iodo, and is preferably chloro or fluoro, more preferably fluoro.

In one embodiment, the carboaryl radical is preferably a phenyl radical, which may be monosubstituted, for example, by a propargylamino or methoxy group, preferably at position 4, or it is polysubstituted, wherein the substituents are preferably halogen atoms, more preferably chloro or fluoro, sulfo, propargylamino, alkoxy, aminoalkylamino, and trialkylammonium. In one embodiment, the phenyl is disubstituted by chloro or fluoro. In more preferred embodiments, the phenyl is pentasubstituted, wherein the substituents are preferably 5 fluoro atoms, or 4 fluoro atoms and a substituent selected from alkoxy, propargylamino, aminoalkylamino, and trialkylammonium.

As used herein, the term “heteroaryl”, by itself or as part of the mixed carboaryl-heteroaryl radical, refers to a 5-6 membered aromatic ring containing 1-3 heteroatoms selected from O, S and N such as, but not limited to, pyrrolyl, furyl, thienyl, imidazolyl, pirazolyl, oxazolyl, thiazolyl, pyridyl, pirazinyl, pyrimidinyl, 1,3,4-triazinyl, or 1,2,3-triazinyl, preferably pyridyl, which may be substituted as defined above for the carboaryl radical. When the heteroaryl has a N atom in the ring, it may be substituted at the ring N atom, preferably by an alkyl or alkynyl group as described above. In preferred embodiments, the heteroaryl radical is a N—(C₁-C₈)alkyl-pyridylium, preferably 2-, 3- or 4-(N-methyl)pyridylium, or N-propargyl-pyridylium.

The term “mixed carboaryl-heteroaryl” refers to a radical derived from a carboaryl and a heteroaryl radical condensed to each other such as. benzofuryl, isobenzofuryl, indolyl, benzimidazolyl, benzthiazolyl, benzoxazolyl, quinoline, isoquinoline and the like, or covalently linked to each other such as pyridilium-phenyl. The mixed carboaryl-heteroaryl radical may be substituted as defined above. It is to be understood that the substitutions may be in any of the carbocyclic and/or heterocyclic rings.

Examples of mixed carboaryl-heteroaryl radical that can be used according to the invention include N—(C₁-C₈)alkyl-pyridylium-tetrafluorophenyl, for example, 4-(N-methyl-2-pyridylium)-2,3,5,6-tetrafluoro-phenyl and the corresponding 3- and 4-(N-methyl) pyridylium compounds.

The saturated 5-6 membered ring optionally containing a further heteroatom selected from O, S and N may be preferably tetrahydropyrrolyl, piperidinyl, morpholino, thiomorpholino, and piperazino, which may be further substituted at the second N atom by C₁-C₄ alkyl, hydroxyalkyl or benzyl.

In some preferred embodiments, the carboaryl is 2,6-dichlorophenyl, 2,6-difluorophenyl, 4-sulfophenyl, 4-methoxyphenyl, pentafluorophenyl, 4-methoxy-2,3,5,6-tetrafluorophenyl, 4-N-propargylamino-2,3,5,6-tetrafluorophenyl, or 4-N-propargylamino-phenyl; the heteroaryl is 4-(N-methyl)-pyridylium, 2-(N-methyl)-pyridylium, 4-(N-propargyl)-pyridylium, or 2-(N-propargyl)-pyridylium, and the carboaryl-heteroaryl is 4-(pyridyl)-2,3,5,6-tetrafluorophenyl, 4-(N-methyl-pyridylium)-2,3,5,6-tetrafluorophenyl, 4-(N-propargyl-pyridylium)-2,3,5,6-tetra-fluorophenyl, 2-(N-propargyl-pyridylium)-2,3,5,6-tetrafluorophenyl.

In some more preferred embodiments, Ar₁, Ar₂ and Ar₃ are the same and each is CF₃, 4-sulpho-phenyl, pentafluorophenyl, 4-methoxy-2,3,5,6-tetrafluoro-phenyl, 4-(N-methyl)-pyridylium, 2-(N-methyl)-pyridylium, 4-(N-propargyl)-pyridylium, or 2-(N-propargyl)-pyridylium.

In some other more preferred embodiments, Ar₁ and Ar₃ are 4-(N-methyl)-pyridylium and Ar₂ is pentafluorophenyl; or Ar₁ and Ar₃ are 4-N-propargylamino-2,3,5,6-tetrafluorophenyl and Ar₂ is 4-methoxyphenyl; or Ar₁ and Ar₃ are 4-(N-propargyl)-pyridylium and Ar₂ is pentafluorophenyl; or Ar₁ and Ar₃ are 2-(N-propargyl)-pyridylium and Ar₂ is pentafluorophenyl; or Ar₁ and Ar₃ are 2-(N-methyl)-pyridylium and Ar₂ is 4-N-propargylaminophenyl.

In some preferred embodiments, E₂ and E₁₇ are the same and are H, SO₃H, SO₂NH-propargyl or SO₂NH—CH₂—COOH.

The present invention further provides new corroles carrying one or more radicals substituted by a propargylamino group or one or more nitrogen-containing heteroaryl radicals substituted by propargyl at the ring N atom.

In particular, the invention relates to metal-chelated corroles of the formula I above carrying a propargyl group, wherein either: (i) at least one of Ar₂, Ar₂ and Ar₃ is a carboaryl radical substituted by a —NR₁R₂ group, wherein R1 is H and R2 is propargyl, or a N-heteroaryl radical substituted at the ring N atom by propargyl; or (ii) at least one of E2 and E17 is —CONR1R2 or —SO2N—R1R2, wherein R1 is H and R2 is propargyl.

In some preferred embodiments, in the new corroles of formula I, E₂ and E₁₇ each is SO₂N—R₁R₂, wherein R₁ is H and R₂ is propargyl, or at position 5, 10 and/or 15, Ar₁, Ar₂ and/or Ar₃ is a phenyl radical solely substituted by a propargylamino group, preferably at position 4, or by a propargylamino group and other substituents such as halogen, preferably fluoro, or Ar₁, Ar₂ and/or Ar₃ is a pyridyl radical substituted by propargyl at the ring N atom.

Examples of propargyl-containing groups according to the invention include, but are not limited to, 4-(N-propargyl)-pyridylium, 2-(N-propargyl)-pyridylium, 4-N-propargylaminophenyl and 4-N-propargylamino-2,3,5,6-tetrafluorophenyl at positions 5, 10 and/or 15 of the corrole or —SO₂—NH-propargyl at positions 2 and 17 of the corrole.

In preferred embodiments, the propargyl-containing corrole is metalated and the central atom is more preferably Fe or Mn.

In some preferred embodiments, the corrole for use in the present invention is selected from:

(i) the corroles in which E₂ and E₁₇ are both SO₃H, Ar₁, Ar₂ and Ar₃ each is pentafluorophenyl, and M is Fe (herein designated corrole 1C-Fe), Mn (herein designated corrole 1C-Mn), or Cu (herein designated corrole 1C-Cu); or Ar₁, Ar₂ and Ar₃ each is CF₃ and M is Fe or Mn;

(ii) the corrole in which E₂ and E₁₇ are both SO₃H, Ar₁, Ar₂ and Ar₃ each is 4-methoxy-2,3,5,6,-tetrafluorophenyl, and M is Fe (herein designated corrole 6C-Fe);

(iii) the corrole in which E₂ and E₁₇ are both SO₂NH—CH₂—COOH, Ar₁, Ar₂ and Ar₃ each is pentafluorophenyl, and M is Fe (herein designated corrole 5C-Fe);

(iv) the corrole in which E₂ and E₁₇ are both H, Ar₁ and Ar₃ each is 4-(N-methyl)-pyridylium, Ar₂ is pentafluorophenyl, and M is Mn (herein designated corrole 2C-Mn);

(v) the corrole in which E₂ and E₁₇ are both H, Ar₁, Ar₂ and Ar₃ each is 2-(N-methyl)-pyridylium, and M is Mn (herein designated corrole 3C-Mn);

(vi) the corrole in which E₂ and E₁₇ are both H, Ar₁ and Ar₃ each is 2-(N-methyl)-pyridylium, Ar₂ is pentafluorophenyl, and M is Mn (herein designated corrole 4C-Mn); and

(vii) the corroles in which E₂ and E₁₇ are both SO₃H, Ar₁, Ar₂ and Ar₃ each is CF₃, and M is Mn or Fe (herein designated corrole M-Mn or H-Fe, respectively);

(viii) the corroles in which E₂ and E₁₇ are both H, Ar₁ and Ar₃ each is 4-(N-propargyl)-pyridylium, Ar₂ is pentafluorophenyl and M is Mn or Fe (herein designated corrole E-pr-Mn or E-pr-Fe, respectively);

(ix) the corrole in which E₂ and E₁₇ are both H, Ar₁ and Ar₃ each is 2-(N-propargyl)-pyridylium, Ar₂ is pentafluorophenyl, and M is Mn or Fe (herein designated corrole H-Mn or H-Fe, respectively);

(x) the corroles in which E₂ and E₁₇ are both SO₃H, Ar₁ and Ar₃ each is 4-N-propargylamino-2,3,5,6-tetrafluorophenyl and Ar₂ is 4-methoxyphenyl, and M is Mn or Fe (herein designated corrole I-Mn or I-Fe, respectively);

(xi) the corrole in which E₂ and E₁₇ are both H, Ar₁ and Ar₃ each is 2-(N-methyl)-pyridylium, Ar₂ is 4-propargylamino-phenyl, and M is Mn or Fe (herein designated corrole J-Mn or J-Fe, respectively);

(xii) the corroles in which E₂ and E₁₇ are both —SO₂—NH-propargyl, Ar₁, Ar₂ and Ar₃ each is pentafluorophenyl, and M is Mn or Fe (herein designated corrole K-Mn or K-Fe, respectively); and

(xiii) the corroles in which E₂ and E₁₇ are both —SO₂—NH-propargyl, Ar₁, Ar₂ and Arₐ each is CF₃, and M is Mn or Fe (herein designated corrole M-Mn or M-Fe, respectively).

The structural formulas of the above-mentioned corroles (i)-(vii) and propargyl-containing corroles (viii)-(xiii) are depicted in Schemes 1 and 2, respectively, herein at the end of the description, just before the references.

In more preferred embodiments, the corroles for use in the present invention are the corroles 1C-Fe, 1C-Mn, 2C-Mn, 3C-Mn, 4C-Mn, 5C-Fe, 5C-Fe, and E-pr-Mn.

Some of the corroles not containing a propargyl group have been disclosed in applications WO 03/004021 and PCT/IL2008/001066 of the same applicant, which are herewith incorporated by reference as if fully disclosed herein.

Also contemplated by the present invention are pharmaceutically acceptable salts of the corrole of formula I.

Pharmaceutically acceptable salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine (see, for example, Berge S. M., et al., “Pharmaceutical Salts,” (1977) J. of Pharmaceutical Science, 66:1-19). The salts can also be pharmaceutically acceptable quaternary salts such as a quaternary salt of the formula —NRR′R″+Z′ wherein R, R′ and R″ each is independently hydrogen, alkyl or benzyl and Z is a counterion, including chloride, bromide, iodide, O-alkyl, toluenesulfonate, methylsulfonate, sulfonate, phosphate, or carboxylate.

Pharmaceutically acceptable acid addition salts of the compounds include salts derived from inorganic acids such as hydrochloric, nitric, phosphoric, sulfuric, hydrobromic, hydriodic, phosphorous, and the like, as well as salts derived from organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, trifluoromethyl sulfonate or tosylate and the like. Also contemplated are salts of amino acids such as arginate and the like and gluconate or galacturonate (see, for example, Berge S. M., et al., “Pharmaceutical Salts,” (1977) J. of Pharmaceutical Science, 66:1-19).

The present invention further provides a pharmaceutical composition comprising a new propargylamino-substituted corrole, a pharmaceutically acceptable salt or an optical isomer thereof and a pharmaceutically acceptable carrier.

The invention still further relates to a pharmaceutical composition for neuroprotection and neurorescue, particularly for treatment of diabetes and neurodegenerative diseases, disorders or conditions, comprising a pharmaceutically acceptable carrier and a metal complex of an amphiphilic corrole, an optical isomer or a pharmaceutically acceptable salt thereof, preferably of formula I herein.

Also provided by the present invention is a method for treatment of diabetes or a neurodegenerative disease, disorder or condition, comprising administering to an individual in need a metal complex of an amphiphilic corrole, an optical isomer or a pharmaceutically acceptable salt thereof, preferably of formula I herein.

As used herein, the term “treatment” means alleviating or ameliorating the symptoms or complications of diabetes or of the neurodegenerative disease, disorder or consition, preventing their progress or curing. The corrole according to the patient is therefore administered to the patient in an effective amount as many times as necessary to achieve one or more of these goals.

The pharmaceutical compositions of the present invention comprising metal complexes of corroles are formulated for administration to the patient using techniques well-known in the art, for example, as summarized in Remington's Pharmaceutical Sciences, Mack Publishing Co., Easton, Penna., latest edition.

In a preferred embodiment, the pharmaceutical composition for use in the present invention is administered parenterally, for example, by intravenous or intramuscular injection, or preferably orally. The doses will depend on the type of disease or disorder and condition and age of the patient and may vary between 0.1 to 10 mg/kg/day.

The corroles according to the invention, including the novel propargyl-containing corroles, are useful as neuroprotective and neurorescuing agents for treatment and/or prevention of diabetes and neurodegenerative diseases, disorders or conditions and any other disease, disorder or condition that can benefit from the neuroprotection and neurorescue activity provided by the corroles.

In one embodiment, the disease, disorder or condition that can be treated by these corroles is diabetes type II and complications thereof.

In another embodiment, the disease, disorder or condition is a neurodegenerative disease, disorder or condition including Parkinson's disease (PD), Alzheimer's disease (AD), Huntington disease (HD), amyotrophic lateral sclerosis (ALS), multiple sclerosis, Friedreich's ataxia, Hallervorden-Spatz disease, a dementia, and psychiatric diseases, disorders and conditions;

The dementia may be an AD or a non-AD dementia such as Lewy body dementia, vascular dementia and a dementia caused by Parkinson's disease, Huntington's disease, Creutzfeldt-Jakob disease, head trauma or HIV infection. The psychiatric disease, disorder or condition may be an affective or mood disorder including depression, a dysthymic disorder, a bipolar disorder, a cyclothymic disorder, schizophrenia or a schizophrenia-related disorder such as brief psychotic disorder, a schizophreniform disorder, a schizoaffective disorder and delusional disorder, or it may be drug use and dependence such as alcoholism, opiate dependence, cocaine dependence, amphetamine dependence, hallucinogen dependence, or phencyclidine use and withdrawal symptoms related thereto.

In another embodiment, the disease, disorder or condition is a memory loss disorder such as amnesia or memory loss associated with Alzheimer's type dementia, with a non-Alzheimer's type dementia, or with a disease or disorder selected from Parkinson's disease, Huntington's disease, Creutzfeldt-Jakob disease, head trauma, HIV infection, hypothyroidism and vitamin B 12 deficiency;

In further embodiments, the disease, disorder or condition is an acute neurological traumatic disorder or neurotrauma such as head trauma injury or spinal cord trauma injury; a) demyelinating disease such as multiple sclerosis; a seizure disorder such as epilepsy; a cerebrovascular disorder such as brain ischemia or stroke; a behavior disorder of neurological origin that may be a hyperactive syndrome or an attention deficit disorder; and a neurotoxic injury which is caused by a neurotoxin such as a nerve gas or the toxin delivery system of poisonous snakes, fish or animals.

In some preferred embodiments, the neurodegenerative disease is Parkinson's disease, Huntington's disease, Alzheimer's disease or amyotrophic lateral sclerosis.

As shown in the examples herein, the corroles metal complexes, preferably the Fe and Mn complexes, are highly potent in protecting the activity of insulin-secreting cell lines against the toxic effects of additives that mimic naturally occurring oxidative/nitrosative stress. A comparison with structurally related metalloporphyrins reveals that the corrole derivatives are significantly superior. Even more remarkable is the observed neurorescue/neurorestorative effect in the models of neurodegenerative diseases (AD, PD and ALS) against oxidative/nitrosative stress and deprivation of neurotrophic factor support.

It is also shown herein in the examples that the corroles inhibit the monoamine oxidase enzymes MAO-A and MAO-B.

The corroles used herein display a consistent superiority in all monitored neuroprotection and neurorescue parameters. It is also shown that the corroles have a positive impact in cell lines model for diabetes and neurodegenerative diseases to retard or perhaps even reverse the accelerated rate of cytodegeneration. It is also shown that some of the corroles used herein may cross the BBB, likely due to the strong and spontaneous association to proteins that may facilitate the process.

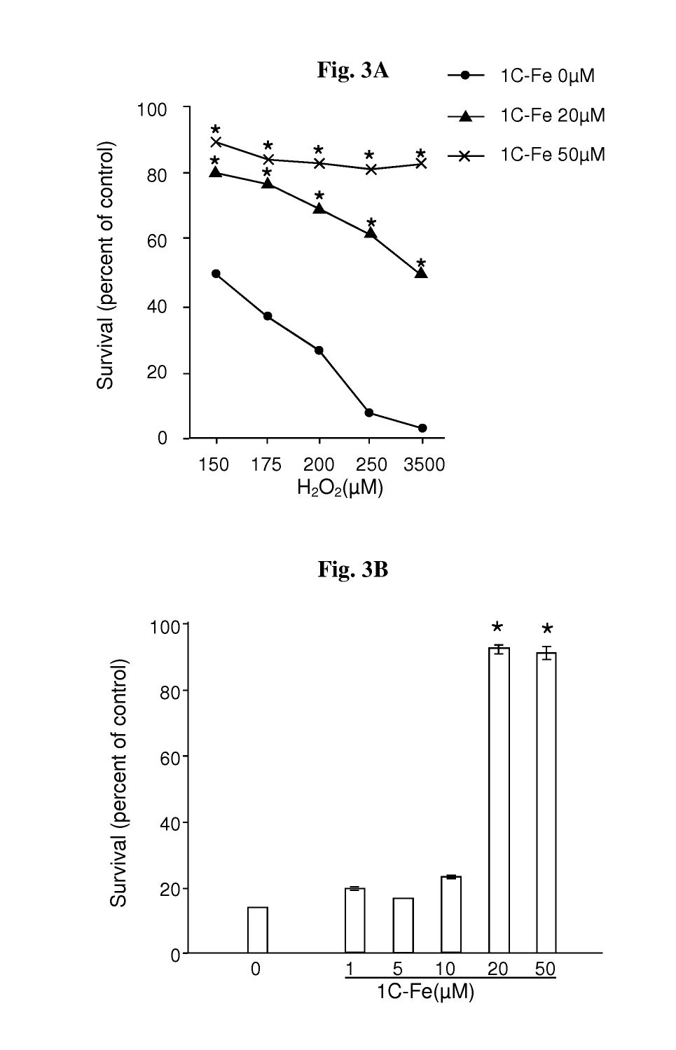

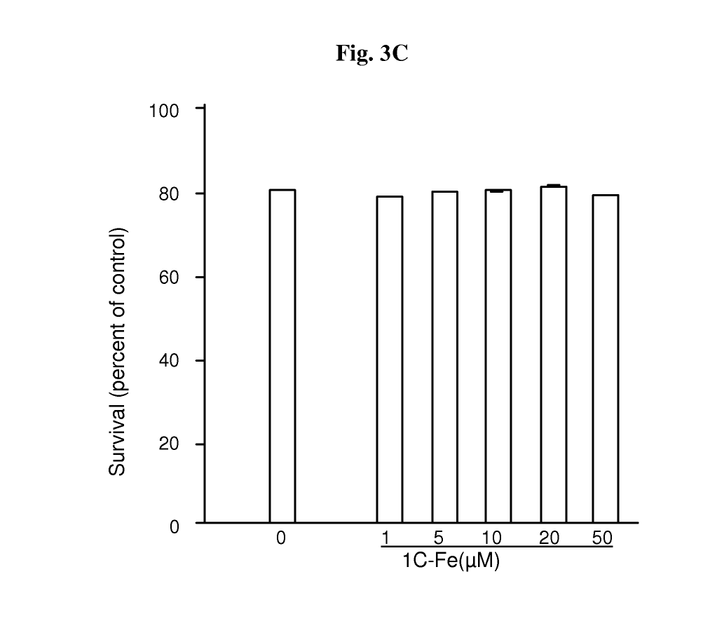

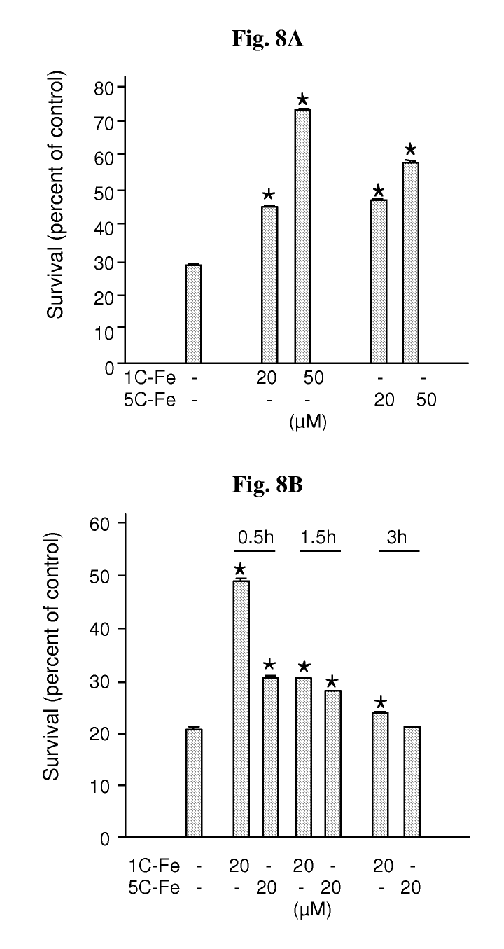

The results herein indicate that the corroles are highly potent regarding the protective activity of insulin-secreting cells against the toxic effects of additives that mimic naturally occurring oxidative/nitrosative stress (FIGS. 3-7). Even more remarkable is the observed neurorescue/neurorestorative effect in the models of neurodegenerative diseases (AD, PD and ALS) against oxidative/nitrosative stress and deprivation of neurotrophic factor support (FIGS. 8-20). Consistently, iron corroles 1C-Fe and 5C-Fe and manganese corroles 2C-Mn and 3C-Mn displayed superiority over other corroles.

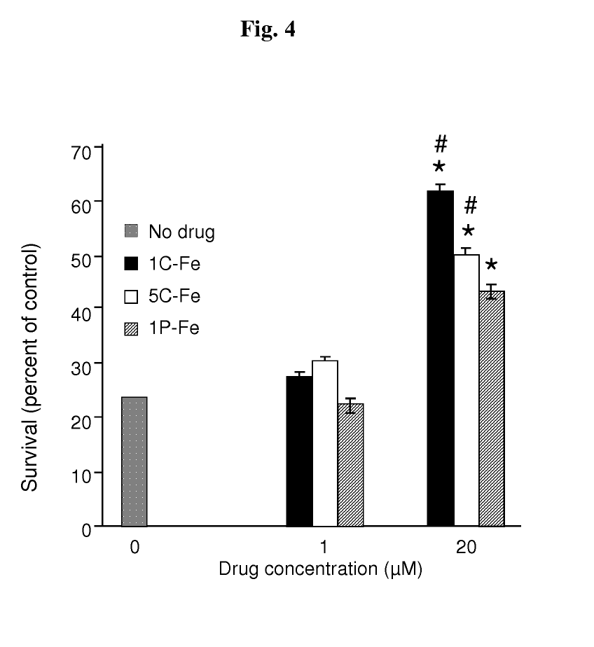

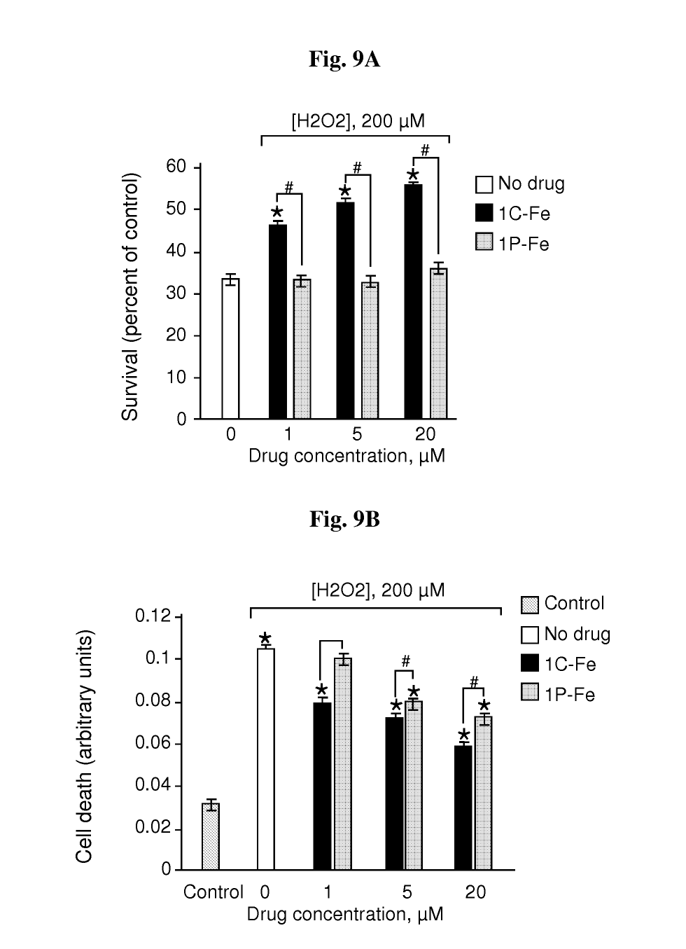

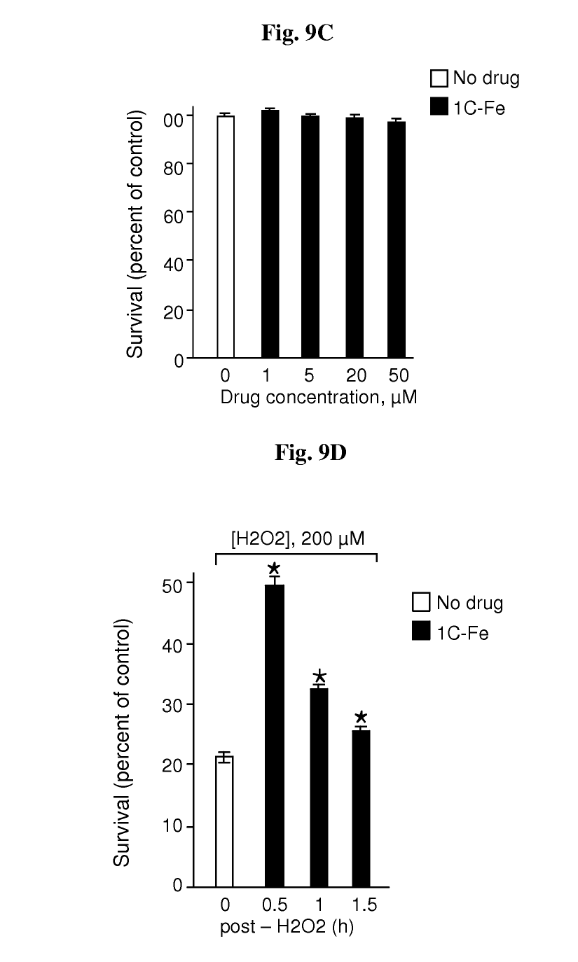

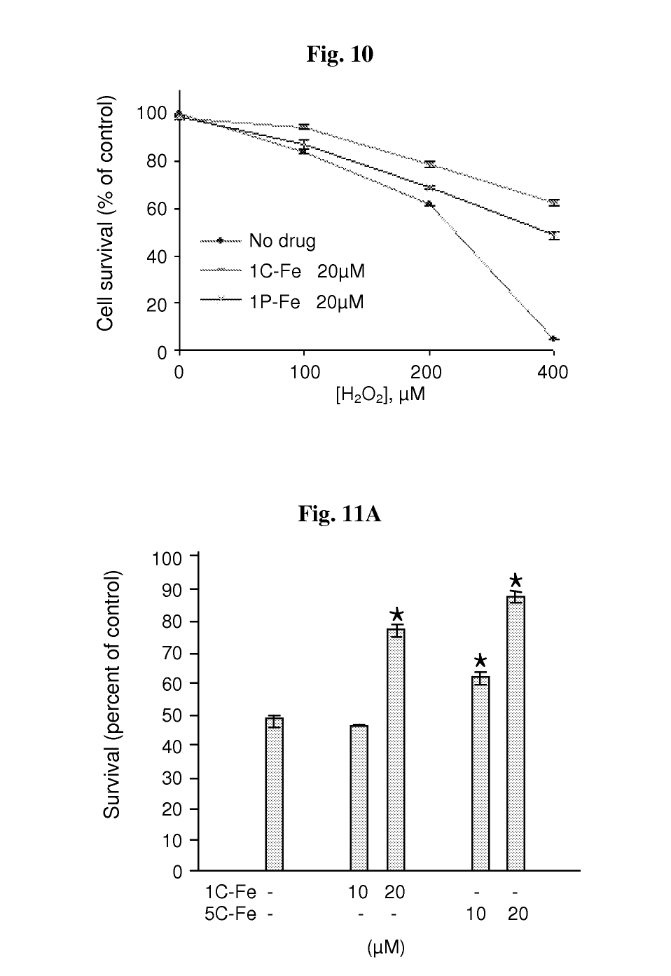

The present findings suggest that corroles 1C-Fe, 5C-Fe, 2C-Mn and 3C-Mn may have a positive impact on diabetes, aging and neurodegenerative diseases as to retard, or perhaps, even reverse the accelerated rate of cytodegeneration. This may suggest a potential disease modifying activity. It is further important to emphasize the observation that the corrole complexes perform significantly better than structurally analogous porphyrins (1C-Fe vs. 1P-Fe and 2C-Mn vs. 2P-Mn) in many cases (see FIGS. 4, 7, 9, 12, 15 and 18). Notably, the iron corrole 1C-Fe and the pyridinium-substituted manganese corrole 2C-Mn were successful against SIN-induced toxicity in RIN-m cells, while the sulfonato-substituted manganese corrole 1C-Mn was ineffective. This correlates very well with the results of Table 3, suggesting that the rate of detoxification by 1C-Mn and its SOD activity are apparently too low. In addition, the observation that corroles 1C-Fe and 5C-Fe were able not only to protect but also to rescue neurons when administered after the oxidative damage was induced (FIGS. 8B, 9D, 11B, 14B and 15C), suggests: a) that the compounds have the ability to enter the cells and b) that mechanisms other than a direct antioxidant effect contribute to their neuroprotective action. A detailed toxicity study has not been performed, nevertheless the corrole compounds tested so far do not display in vitro cytotoxicity up to 50 μM (see: FIGS. 3 and 9C), and mice that received 10 mg/kg amounts of corroles 1C-Fe and 1C-Mn for 10 weeks did not display any observable abnormal behaviour or weight loss.

The invention will now be illustrated by the following non-limiting examples.

### EXAMPLES

In the examples, the following compounds will be identified by abbreviated names as detailed below. The chemical structure of these compounds is presented in Schemes 1 and 2, just before the References.

Corrole 1C-Fe: 2,17-bis(sulfonato)-5,10,15-tris(pentafluorophenyl)-corrolato iron(III)

Corrole 1C-Mn: 2,17-bis(sulfonato)-5,10,15-tris(pentafluorophenyl)-corrolato manganese(III)

Corrole 1C-Cu: 2,17-bis(sulfonato)-5,10,15-tris(pentafluorophenyl)-corrolato copper(III)

Corrole 1C-Ga: 2,17-bis(sulfonato)-5,10,15-tris(pentafluorophenyl)-corrolato galium(III)

Corrole 2C-Mn: 5,15-bis(4-N-methyl-pyridilium)-10-pentafluorophenyl-corrolato manganese(III) diiodide salt

Corrole 3C-Mn: 5,10,15-tris(N-methyl-2-pyridilium)corrolato manganese(III)

Corrole 4C-Mn: 5,15-bis(N-methyl-2-pyridilium) 10-pentafluorophenyl corrolato manganese(III)

Corrole 5C-Fe: 2,17-bis(N-sulfonylglycine)-5,10,15-tris(pentafluoro-phenyl)-corrolato iron(III)

Corrole 6C-IFe: 5,10,15 tris(4-methoxy-tetrafluorophenyl)-corrolato iron (III).

Corrole E-pr-Mn: 5,15-bis(4-N-propargyl-pyridilium)-10-pentafluorophenyl corrolato manganese (III)

Porphyrin 1P-Fe: 5,10,15,20-tetra(4-sulfonatophenyl)-porphyrinato iron(III)

Porphyrin 2P-Mn: 5,15-bis(N-methyl-4-pyridilium)-10,20-bis-(pentafluoro-phenyl)porphyrinato manganese(III) diiodide salt The corroles 1C-Fe, 1C-Mn, 1C-Cu and 1C-Ga were prepared according to procedures previously disclosed by the inventors (WO 03/004021, U.S. Pat. No. 6,939,963; Mahammed and Gross, 2006; Saltsman et al., 2002). The corrole 2C-Mn was prepared as described previously by the inventors (Gershman et al., 2007). The corroles 3C-Mn, 4C-Mn, 5C-Fe and 6C-Fe as well the propargyl-substituted corroles are new and their synthesis is described hereinbelow in the Examples. In the syntheses, the solvents and standard chemicals were purchased from reliable sources and used as received.

### Example 1

### Catalytic Decomposition of ROS/RNS by the Metal Corroles

SOD activity (enzyme-like catalytic decomposition of superoxide anion radical) of the corroles 1C-Fe, 6C-Fe, 1C-Mn, 1C-Cu, 2C-Mn and 3C-Mn was examined via the cytochrome C assay. The catalytic rate constants for decomposition of peroxynitrite (PN) by the newly prepared iron corrole 6C-Fe and manganese corrole 3C-Mn were determined via stopped-flow kinetics as previously described for corroles 1C-Fe, 1C-Mn, and 2C-Mn (Mahammed and Gross, 2006; Gershman et al., 2007). These tests are performed in order to estimate how electronic and structural variables affect reactivity, which is a crucial factor for design of new complexes.

TABLE 1
SOD activity and catalytic rate constants for decomposition of PN.^{a}

| Complex | 1C—Fe | 6C—Fe | 1C—Mn | 1C—Cu | 2C—Mn | 3C—Mn |
|---|---|---|---|---|---|---|
| IC₅₀ (μM) | 1.64 | 1.18 | 5.9 | 12 | 1.52 | 3.27 |
| k_{cat} (M⁻¹s⁻¹) | 2.0 × 10⁶ | n.d. | 4.0 × 10⁴ | n.d. | 4.0 × 10⁵ | 2.5 × 10⁴ |

n.d. = not yet determined.
^{a}Note how IC₅₀ and k_{cat} are affected by the kind of metal ion (1C—Fe < 1C—Mn << 1C—Cu) and by the corrole substituents in both the Mn and iron complexes.

The results presented in Table 1 show that corroles display substantial SOD-like activity in the cytochrome C assay and also suggest that the IC₅₀ values may be further reduced by increasing the electron-richness of the complexes (compare 6C-Fe with 1C-Fe for example). This can be done, for example, by using corroles with much more electron-donating aryl groups and determination of their electrochemical redox potential. We have already confirmed that in a series of corroles that are not water-soluble (work performed in water/DMSO mixtures), shifting of redox potentials and SOD activities are strongly correlated indeed (not shown).

The catalase-like activity (catalytic decomposition of hydrogen peroxide into molecular oxygen and water) of the metalated corroles was also examined (by an oxygen-measuring electrode), revealing that the iron corroles decompose H₂O₂ much faster than the corresponding manganese complexes.

In all these investigations, even for very large numbers of catalytic turnovers, there was no oxidative degradation of the corrole complexes.

### Example 2

### Determination of Peroxynitrite Decomposition Rates by New Manganese Corroles

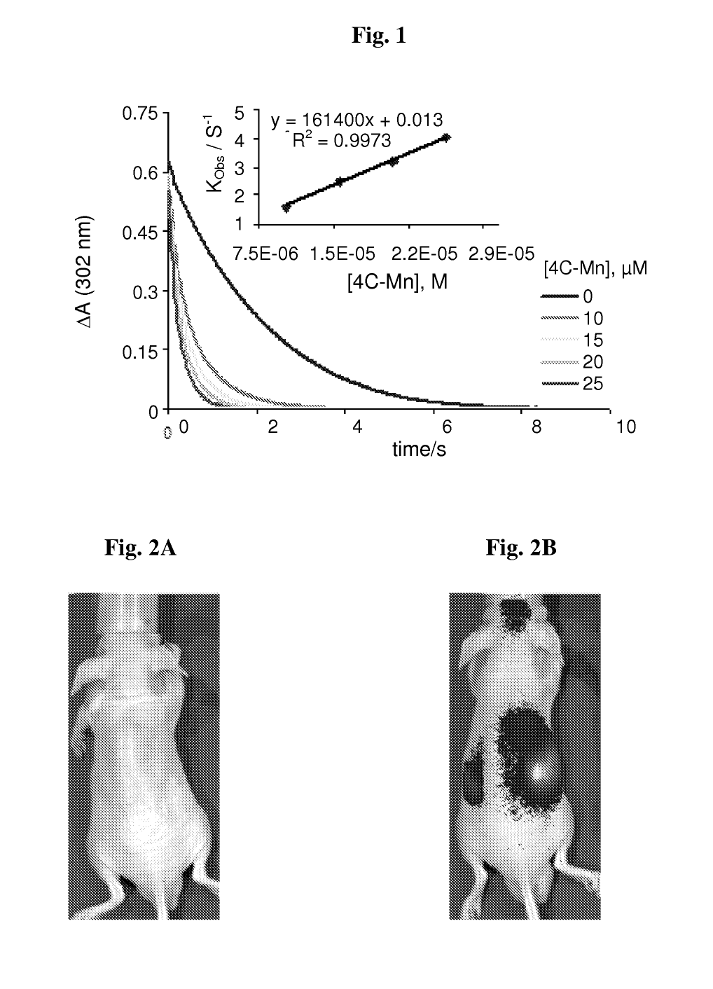

The investigations were performed by examining the decay of 385 μM peroxynitrite at pH 7.4 and 25° C. in the presence and absence of various amounts of Mn corrole catalysts 2C-Mn, 2C′-Mn, 2C″-Mn, 3C-Mn and 4C-Mn. The differences between 2C-Mn, 2C′-Mn, and 2C″-Mn is in the identity of the 10-aryl group, which is C₆F₅, para-anisyl, and phenyl, respectively. The results (one example using 4C-Mn is shown in FIG. 1) were obtained by monitoring changes in absorbance at 302 nm, the λₘₐₓ of peroxynitrite. The catalytic performances of five of the above compounds were tested; and the thus elucidated k_{cat} values are presented in Table 2. Importantly, in sharp contrast with the corroles, none of the structurally similar manganese (III) porphyrin complexes affected the decomposition of peroxynitrite by any extent (not shown).

TABLE 2
Peroxynitrite decomposition rate by various manganese corroles.

| Complex | 2C-Mn | 2C′-Mn | 2C″-Mn | 3C-Mn | 4C-Mn |
|---|---|---|---|---|---|
| k_{cat} (M⁻¹s⁻¹) | 1.5 × 10⁵ | 7.7 × 10⁴ | 8.1 × 10⁴ | 2.5 × 10⁴ | 1.6 × 10⁵ |

### Example 3

### Rescuing Molecules from Damage Attributable to the Hydroxyl Radical with Fe and Mn Corroles

Corrole complexes were examined with respect to their efficiency of rescuing DMSO from damage attributed to the hydroxyl radical, formed via the Fenton reaction that also involves superoxide anion radical and hydrogen peroxidedifferent ROS and/or RNS. It also mimics reaction occurring in biological systems that are exposed to metal toxicity, the situation that occurs in diseases caused by brain iron accumulation. The results summarized in Table 3 demonstrate that the iron(III) corrole 1C-Fe is very potent in preventing the oxidative damage and that the newly prepared manganese corroles 2C-Mn and especially 3C-Mn apparently protect better than corrole 1C-Mn against metal-induced oxidation of substrates.

TABLE 3
Inhibitory effect of Fe and Mn corroles on oxidation/nitration of selected molecules

| | | | Yield (%) or amount of product^{a} | | | | |
|---|---|---|---|---|---|---|---|
| ROS/RNS, | | | No | | | | |
| reactive species | Substrate | Product | additive | 1C—Fe | 1C—Mn | 2C—Mn | 3C—Mn |
| CuSO₄/ascorbate | DMSO | Formaldehyde | 44 μM | 0 | 20 μM | 7 μM | 0 |
| CuSO₄/glutathione | DMSO | Formaldehyde | 11 μM | 0 | 7 μM | 5 μM | 0 |

### Example 4

### Biodistribution of Corroles

For obtaining information about in-vivo localization and distribution of the various corroles that have sulfonic acid head groups, in-vivo imaging of corroles in whole animals was performed. Of particular interest was to examine whether the leading corrole compounds unlike most porphyrins are able to cross the blood brain barrier (BBB).

The corrole 1C-Fe is shown herein to be a neurorescue and neuroprotective compound. The corrole 1C-Ga (Scheme 1) is a fluorescent analogue of 1C-Fe that does not exhibit those abilities of 1C-Fe, but can be used as a material that can be easily detected in cells and tissues due to its fluorescence. Because 1C-Fe and 1C-Ga differ only by the metal center, it is assumed that their biodistribution is the same.

The in vivo imaging experiments were performed as follows. Nude mice received i.p. injections of the fluorescent gallium(III) corrole 1C-Ga and were examined by IVIS 200 imaging system [Xenogen]. The result shown in FIG. 2B (30 min after injection of 19 mg/kg 1C-Ga) demonstrates possible BBB penetration and accumulation of the corrole complex in brain area.

### Example 5

### Protective Effect of Iron Corrole 1C-Fe on Clonal Beta Cell Lines Exposed to Hydrogen Peroxide (H₂O₂)-Neurorescue/Neurorestorative Effects

Islets and especially β cells contain among the lowest levels of antioxidant enzyme activities compared to other tissues. These facts are believed to be responsible for the high sensitivity of insulin-producing cells to various insults, leading to destruction of β cells and consequently resulting in diabetes. This triggered our study on examining protective properties of corroles against oxidative stress in insulin-producing β cells.

Two insulin-secreting beta cell lines, rat insulinoma (INS-1E) and RIN-m cells, were used to examine the protective effect of corrole 1C-Fe. The cells were cultured at 37° C., in a humid 5% CO₂, 95% air environment in regular RPMI-1640 medium (Invitrogen) supplemented with 5% fetal calf serum (FCS).

After 2 days of culture, INS-1E cells (5×10⁴ cells/well in 96-well plates) were treated with corrole 1C-Fe (20 and 50 μM) added 30 minutes prior to treatment with increasing concentrations of H₂O₂ and then further incubated for 24 h. The results are shown in FIG. 3A.

After 2 days of culture, RIN-m cells (5×10⁴ cells/well in 96-well plates) were treated with corrole 1C-Fe (1-50 μM) before addition of 35 μM of H₂O₂. Cell viability was evaluated by a colorimetric assay for mitochondrial function estimation using the MTT test as previously described (Gassen et al., 1998) and expressed as percentage of control. The results are shown in FIG. 3B.

Data presented in FIGS. 3A and 3B show a significant protective effect of corrole 1C-Fe on beta cells against a wide range of hydrogen peroxide concentrations. Corrole 1C-Fe shows no toxicity when given alone (without H₂O₂) at concentrations up to 50 μM (FIG. 3C).

### Example 6

### Comparative Effects of Corroles 1C-Fe, 1C-Mn, 5C-Fe and 2C-Mn and Porphyrins 1P-Fe and 1P-Mn Against Toxicity Induced by H₂O₂ in RIN-m Cells

RIN-m cells were grown as described in Example 5. After 3 days of culture, RIN-m cells (2.5×10⁴ cells/well in 96-well plates) were treated with corroles 1C-Fe or 5C-Fe or porphyrin 1P-Fe (1 and 20 μM), added 30 min prior to treatment with 35 μM H₂O₂ and further incubated for 24 h. Cell injury was evaluated by MTT test. The results in FIG. 4 represent the mean±SEM of a representative experiment repeated twice with similar results performed under the same conditions. Corroles 1C-Fe and 5C-Fe (in a concentration of 20 μM) exhibit a greater protective effect against toxicity induced by H₂O₂ in RIN-m cells, relative to their structurally analogous porphyrin 1P-Fe.

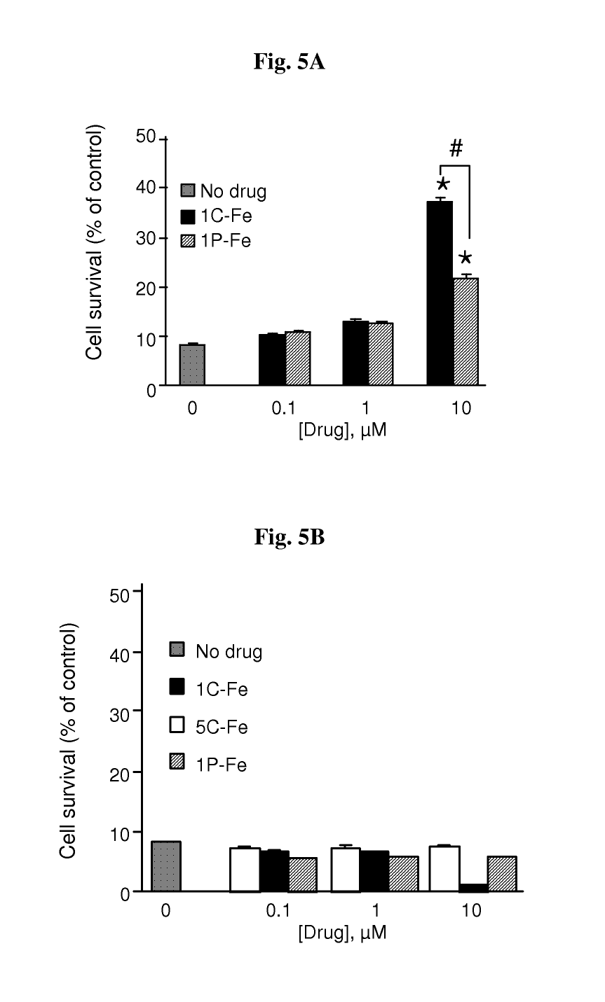

Protective effect of iron(III) corrole complexes against H₂O₂-induced toxicity (150 μM) was compared to that of manganese(III) corroles and related porphyrin. While the iron(III) complexes of corrole 1C-Fe and the structurally related porphyrin 1P-Fe showed cytoprotective effects against H₂O₂, revealing some advantages of the former (FIG. 5A). The Mn(III) complexes 1C-Mn and 2C-Mn and the structurally analogous porphyrin 2P-Mn were totally inefficient (FIGS. 5A-5B).

### Example 7

### Protective Effect of Iron Corroles 1C-Fe and 5C-Fe Against Toxicity Induced by the Nitric Oxide Donor, SIN-1 (a Precursor of Peroxynitrite), in INS-1E and RIN-m Cells

INS-1E cells or RIN-m cells were cultured in RPMI with low serum content (2% FCS), placed in microtiter plates (96 wells) at a density of 2.5×10⁴ cells/well and allowed to attach for 24 h before treatment. Corroles 1C-Fe and 5C-Fe (10 μM) were added 30 min prior to insult with the nitric oxide donor SIN-1 (500 or 600 μM) for a subsequent 24 h period. Cell viability was evaluated by a colorimetric assay for mitochondrial function using 3-(4,5-dimethylthiazol-2-yl)-2,5-diphenyltetrazolium bromide (MTT test), and expressed as percentage of untreated control. Values higher than 100% reflect increased mitochondrial activity induced by the drug. The results represent the mean±SEM of three independent experiments performed under the same conditions.

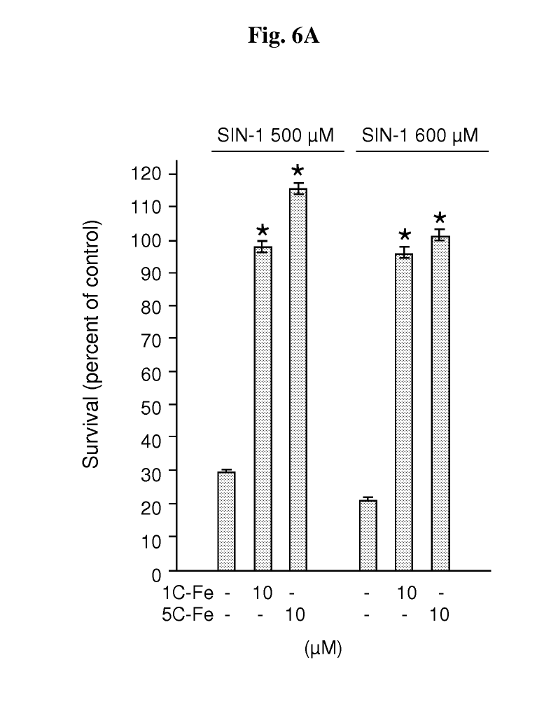

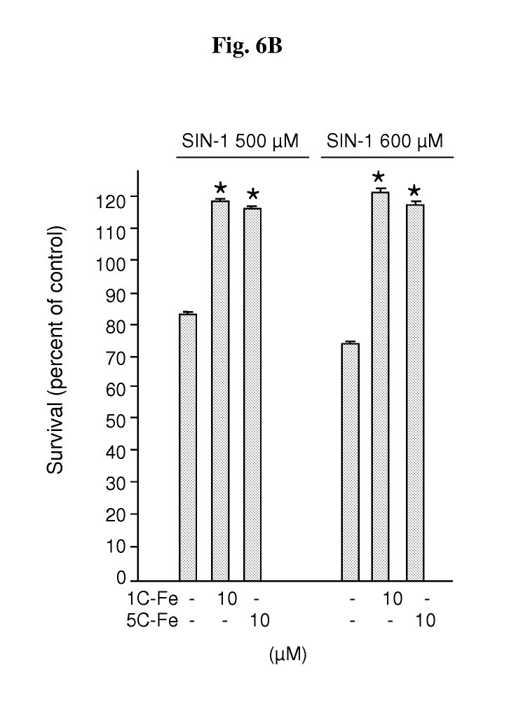

The results depicted in FIGS. 6A-6B demonstrate a marked protective effect of corrole 1C-Fe and 5C-Fe against SIN-1 induced toxicity on INS-1E cells and RIN-m beta cells, respectively. The damage is more pronounced in INS-1E cells, while the protection is extremely efficient in both cases.

### Example 8

### Comparative Effects of Iron Corroles 1C-Fe and Manganese Corroles 1C-Mn and 2C-Mn Relative to their Respective Porphyrins 1P-Fe and 2C-Mn in RIN-m Beta Cells Exposed to SIN-1

Insulin-secreting RIN-m cells were incubated in RPMI with low serum content (2% FCS) and placed in microtiter plates (96 wells) at a density of 2.5×10⁴ cells/well and allowed to attach for 24 h before treatment. Cells were treated with corroles 1C-Fe, 1C-Mn or 2C-Mn or porphyrins 1P-Fe or 2P-Mn, in various concentrations (0.1-20 μM), for 30 min prior to insult with SIN-1 (800 μM) for a subsequent 24 h period. Cell viability was evaluated by MTT test and expressed as percentage of untreated control.

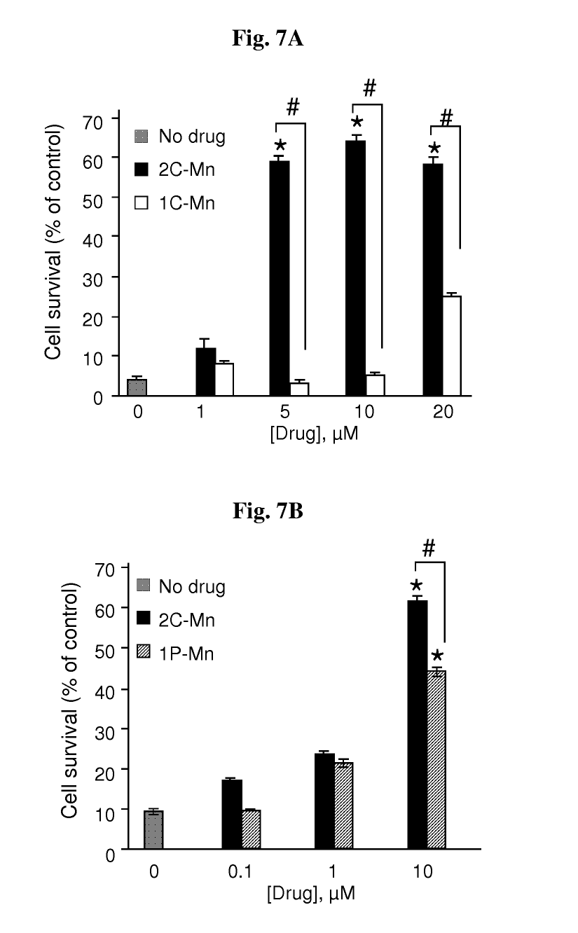

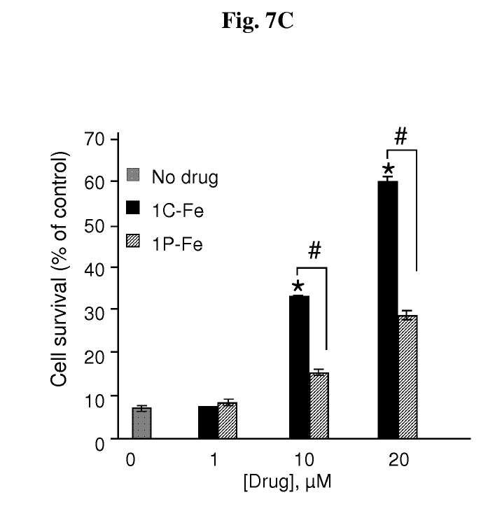

The first comparison concerned the cytoprotective properties of positively charged manganese corrole 2C-Mn vs. its negatively charged analog 1C-Mn (FIG. 7A). The results were unequivocally in favor of the former and 2C-Mn was chosen as leading compound for manganese complexes. For revealing possible corrole's advantages, a structurally analogous porphyrin 2P-Mn was also synthesized and was investigated in the same experiments (FIG. 7C). Iron corrole 1C-Fe and related porphyrin 1P-Fe were also tested under the same conditions, revealing advantages of the former (FIG. 7B).

At each drug concentration, the iron corrole 1C-Fe exhibits a greater protective effect on RIN-m beta cells, against toxicity induced by SIN-1, relative to its respective porphyrin 1P-Fe (FIG. 7B). The Mn-Corrole 2C-Mn also shows improved protective effect on RIN-m cells against toxicity induced by SIN-1, relative to its corresponding porphyrin 2P-Mn (FIG. 7C).

### Example 9

### Neuroprotective and Neurorescue Effects of Iron(III) Corroles 1C-Fe And 5C-Fe on H₂O₂-Induced Cell Death in Human Neuroblastoma SH-SY5Y Cell Line

Human neuroblastoma cell line was also examined under conditions similar to RIN-m cells to reveal the corrole's ability to prevent neuronal death caused by oxidative stress. SH-SY5Y cells were plated in microtiter plates (96 wells) in DMEM-Eagle/F-12(HAM) (1:1), containing 10% FCS. To examine neuroprotective effect, SH-SY5Y cells were treated for 30 min with corroles 1C-Fe and 5C-Fe (20 and 50 μM) before addition of 20 μM H₂O₂ for 24 h. To examine neurorescue, SH-SY5Y cells were first exposed to 20 μM H₂O₂ for 0.5 h, 1.5 h or 3 h, followed by the addition of corroles 1C-Fe and 5C-Fe (20 μM) and incubation for 24 h.

The results are shown in FIGS. 8A-8B. Both corrole 1C-Fe and 5C-Fe show a dose-dependent neuroprotective effect against H₂O₂-induced toxicity in SH-SY5Y cells (FIG. 8A). Moreover, corroles 1C-Fe and 5C-Fe instigated neurorescue of SH-SY5Y cells following insult with H₂O₂ for various time periods (FIG. 8B).

### Example 10

### Comparative Effects of Corrole 1C-Fe and Porphyrin 1P-Fe Against Toxicity Induced by H₂O₂ in SH-SY5Y Cells

Compounds 1C-Fe and 1P-Fe (1-50 μM) were added 30 min before insult with H₂O₂ (200 μM), followed by a subsequent 24 h period. The neuroprotective effects of the iron corrole 1C-Fe and its structurally analogous iron porphyrin 1P-Fe, against H₂O₂-induced neurotoxicity were investigated using MTT reduction assay and cell death ELISA, based on the use of mouse monoclonal antibodies to detect free histones and fragmented DNA (F. Hollderieder et al, Biochemica 2002, 1, 25). The results show (FIGS. 9A-9C) that H₂O₂ at 200 μM for 24 h markedly decreased cultured human neuroblastoma SH-SY5Y cell viability when compared with untreated control cells. Pretreating the cells with corrole 1C-Fe (1-20 μM) for 30 min prior to the incubation with 200 μM H₂O₂ for 24 h, dose-dependently increased cell viability, as determined by MTT reduction when compared with H₂O₂-treated cells (FIG. 9A). Corrole 1C-Fe added in the absence of H₂O₂ (up to 50 μM) had no significant effect on the viability of SH-SY5Y (FIG. 9C). Similar results were obtained when neuroprotection was evaluated by ELISA detection assay, demonstrating that corrole 1C-Fe significantly and dose-dependently protected SH-SY5Y cells against H₂O₂ neurotoxicity (FIG. 9B). Consistently, at all concentrations studied, corrole 1C-Fe displayed superiority over porphyrin 1P-Fe in protecting SH-SY5Y cells from H₂O₂-induced neurotoxicity. In light of the observed neuroprotective effect of corrole 1C-Fe, we attempted to determine the length of the delay between H₂O₂ exposure and corrole 1C-Fe treatment that would still afford neuroprotection. H₂O₂ was administered to SH-SY5Y cells and corrole 1C-Fe was then added for 0.5-1.5 h thereafter. The neuroprotective effect of corrole 1C-Fe remained unaltered at 0.5 h post-H₂O₂ insult (FIGS. 9A and 9D). At 1 and 1.5 h post-H₂O₂ insult, corrole 1C-Fe significantly protected SH-SY5Y cells, but the extent of protection gradually decreased (FIG. 9D). Notably, both pyridinium-substituted manganese corroles 2C-Mn and 3C-Mn (up to 50 mM) were not effective at preventing H₂O₂-mediated cytotoxicity (data not shown).

Cytoprotection by iron complexes against different H₂O₂ concentrations is shown in FIG. 10. As in previous cases, the corrole showed superiority over the analogous porphyrin regarding cell survival in conditions of induced oxidative damage by different H₂O₂ concentrations.

### Example 11

### Neuroprotective and Neurorescue Effects of Iron Corroles 1C-Fe And 5C-Fe Against SIN-1-Induced Cell Death in Human Neuroblastoma SH-SY5Y Cell Line

SH-SY5Y cells were cultured in DMEM-Eagle/F-12(HAM) (1:1) containing 10% FCS. Cells were resuspended in medium containing 2% FCS, seeded in microtiter plates (96 wells) and allowed to attach for 24 h before initiation of treatment. To examine neuroprotective effects, SH-SY5Y cells were treated for 30 min with corroles 1C-Fe and 5C-Fe (10 and 20 μM) before addition of 20 μM H₂O₂ for 24 h. To examine neurorescue, SH-SY5Y cells were first exposed to 20 μM H₂O₂ for 0.5 h, 1.5 h or 3 h followed by the addition of corroles 1C-Fe and 5C-Fe (20 μM) and incubation for 24 h.

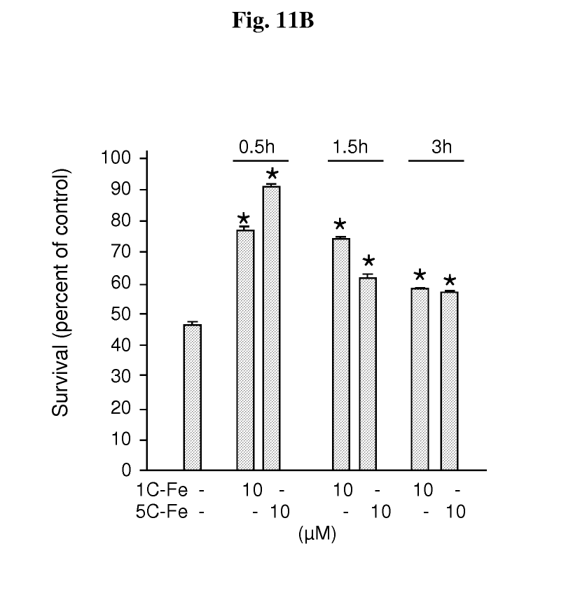

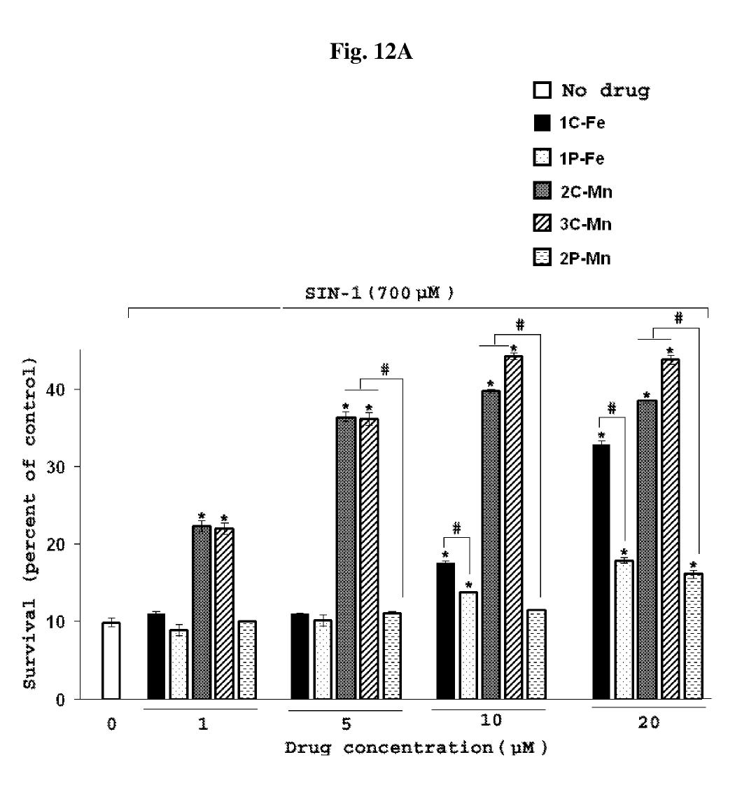

The results are shown in FIGS. 11A-11B. Both corroles 1C-Fe and 5C-Fe showed a dose-dependent neuroprotective effect against SIN-1 induced toxicity in SH-SY5Y cells (FIG. 11A). Moreover, corroles 1C-Fe and 5C-Fe instigated neurorescue of SH-SY5Y cells following insult with SIN-1 for various time periods (FIG. 11B).

### Example 12

### Comparative Effects of Corroles 1C-Fe, 2C-Mn and 3C-Mn and Porphyrins 1P-Fe and 2P-Mn Against Toxicity Induced by SIN-1 in SH-SY5Y Cells

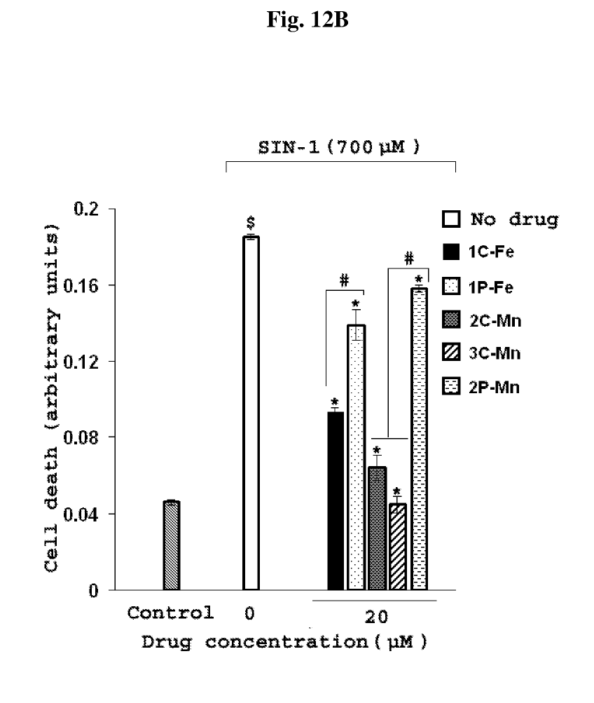

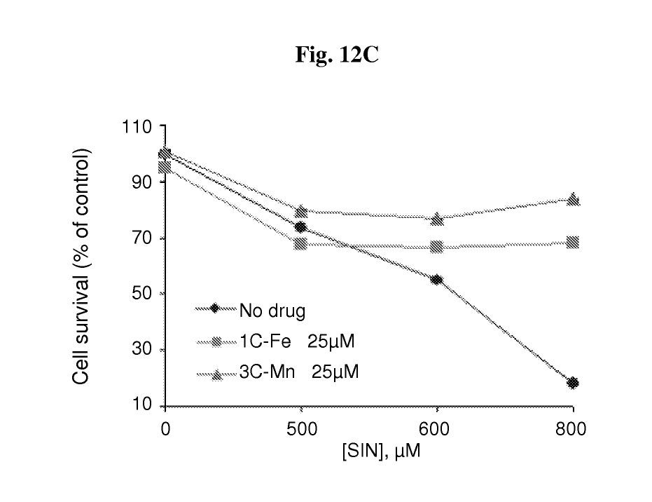

SH-SY5Y cells were pretreated with or without corroles 1C-Fe, 2C-Mn and 3C-Mn and structurally related porphyrins 1P-Fe and 2P-Mn, respectively, 30 min before exposure to SIN-1 for a subsequent 24 h period. FIGS. 12A-12B demonstrate the ability of iron corrole 1C-Fe and manganese corroles 2C-Mn and 3C-Mn to protect, in a dose-dependent manner, SH-SY5Y cells against SIN-1, as determined by both the MTT reduction analysis and ELISA assay. The iron corrole 1C-Fe at concentrations of 10 and 20 μM was able to inhibit neuronal death, but was less potent than the manganese corroles 2C-Mn and 3C-Mn. In addition, both the iron and manganese corroles displayed significant superiority over their respective analogous porphyrins.

In addition to 2C-Mn with its positively-charged para-pyridinium moieties, 3C-Mn with ortho-pyridinium moieties has been examined as well. The manganese(III) corrole 3C-Mn appears to be more effective not only than 2C-Mn, but also shows better cytoprotective properties than the iron(III) corrole complex 1C-Fe. FIG. 12C reveals the ability of both iron(III) and manganese(III) corroles 1C-Fe and 3C-Mn (25 μM) to protect cells against different SIN-1 concentrations, with the cytoprotective properties of 3C-Mn apparently being better than that of 1C-Fe at all toxin concentrations.

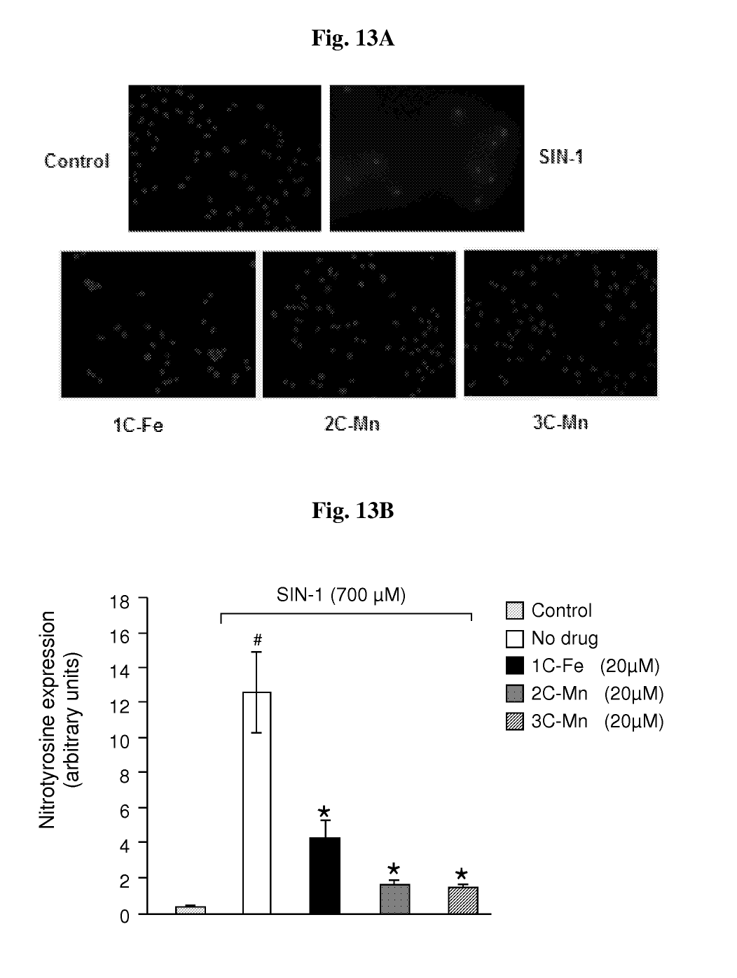

To determine the effect of the corroles on SIN-1-induced cellular protein nitration, SH-SY5Y neuroblastoma cells were treated with SIN-1 (700 μM) in the absence or presence of 20 μM of the corroles 1C-Fe, 2C-Mn, and 3C-Mn. Protein nitration was detected by immunofluorescence analysis using a monoclonal antibody against nitrotyrosine (Ntyr), a biological marker of peroxynitrite. FIGS. 13A-13B illustrate that immunoreactivity to Ntyr was markedly induced by exposure to SIN-1 compared with control cells. Administration of iron corrole resulted in partial (albeit quite remarkable) reduction in Ntyr staining, while both manganese corroles 2C-Mn and 3C-Mn totally abolished SIN-1-induced formation of nitrotyrosine.

### Example 13

### Neuroprotective and Neurorescue Effects of Iron Corroles 1C-Fe and 5C-Fe Against 6-Hydroxydopamine (OHDA)-Induced Cell Death in Human Neuroblastoma SH-SY5Y Cell Line

In this series of experiments, we induced cellular damage by 6-OHDA—as a model for Parkinson's disease. 6-Hydroxydopamine (6-OHDA) is a dopaminergic neurotoxin putatively involved in the pathogenesis of Parkinson's disease (PD). Its neurotoxicity has been related to the production of reactive oxygen species. Under physiological conditions, 6-OHDA is rapidly and nonenzymatically oxidized by molecular oxygen to form hydrogen peroxide and the corresponding p-quinone. The latter then undergoes an intramolecular cyclization followed by a cascade of oxidative reactions resulting in the formation of an insoluble polymeric pigment related to neuromelanin. Although the precise molecular mechanism of cytotoxicity for 6-OHDA remains uncertain, it has been often linked to the production of ROS. The H₂O₂ resulting from the autoxidation of 6-OHDA can easily be reduced in the presence of Fe²⁺ by the Fenton reaction to give the hydroxyl radical, which as mentioned before is considered the most damaging free radical for living cells.

SH-SY5Y cells were cultured in DMEM-Eagle/F-12(HAM) (1:1) containing 10% FCS. Cells were resuspended in medium containing 2% FCS, seeded in microtiter plates (96 wells) and allowed to attach for 24 h. To examine the neuroprotective effect, cells were pretreated for 30 min with corroles 1C-Fe and 5C-Fe (1-20 μM), and were then exposed to 6-OHDA (60 μM) for further 24 h. For neurorescue assessment, SH-SY5Y cells were first treated with 6-OHDA (60 μM) for 0.5 h, 1.5 h, 3 h and 6 h followed by the addition of compounds 1C-Fe and 5C-Fe (10 μM) for further 24 h.

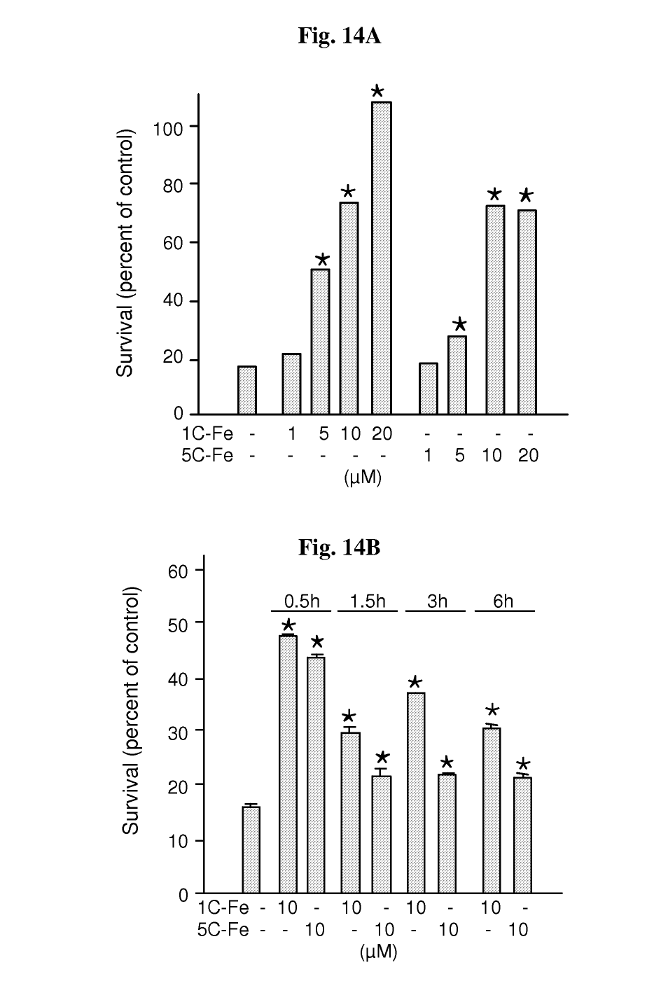

The results are depicted in FIGS. 14A-14B. Both corrole 1C-Fe and 5C-Fe show a dose-dependent neuroprotective effect against 6-OHDA-induced toxicity in SH-SY5Y cells with corrole 1C-Fe showing a greater effect (FIG. 14A). Moreover, corroles 1C-Fe and 5C-Fe instigated neurorescue of SH-SY5Y cells following insult with 6-OHDA for various time periods (FIG. 14B).

### Example 14

### Comparative Effects of Corroles 1C-Fe, 2C-Mn and 3C-Mn and Porphyrins 1P-Fe and 2P-Mn Against 6-OHDA-Induced Neurotoxicity in SH-SY5Y Cells

SH-SY5Y cells were pretreated with or without corroles 1C-Fe, 2C-Mn and 3C-Mn and porphyrins 1P-Fe and 2P-Mn (20 μM), 30 min before exposure to 6-OHDA (40 μM) for a subsequent 24 h period. Cell viability was evaluated by MTT test and cell death was assessed using the apoptotic cell death detection ELISA kit. For neurorescue, SH-SY5Y cells were first exposed to 40 μM 6-OHDA for 0.5. 1, 1.5 and 3 h, followed by the addition of the compounds 1C-Fe, 2C-Mn and 3C-Mn (20 μM) and incubated for further 24 h. Growth-associated protein (GAP-43) was detected by fluorescence microscopy using a specific primary antibody. Apoptotic nuclei were determined by terminal deoxynucleotidyl transferase-mediated UTP-digoxigenin nick end labeling analysis. 4′,6-Diamidino-2-phenylnidole (DAPI) staining was applied for nuclear labeling. 6-OHDA was detected by fluorescence microscopy using a specific primary antibody.

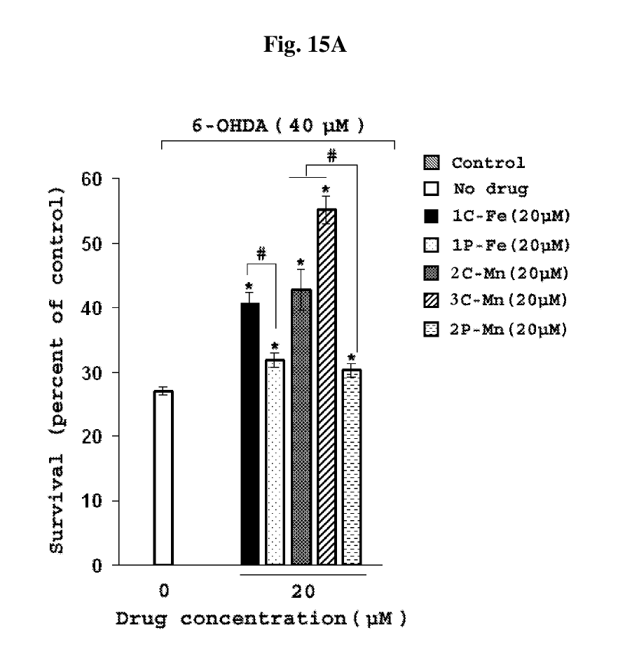

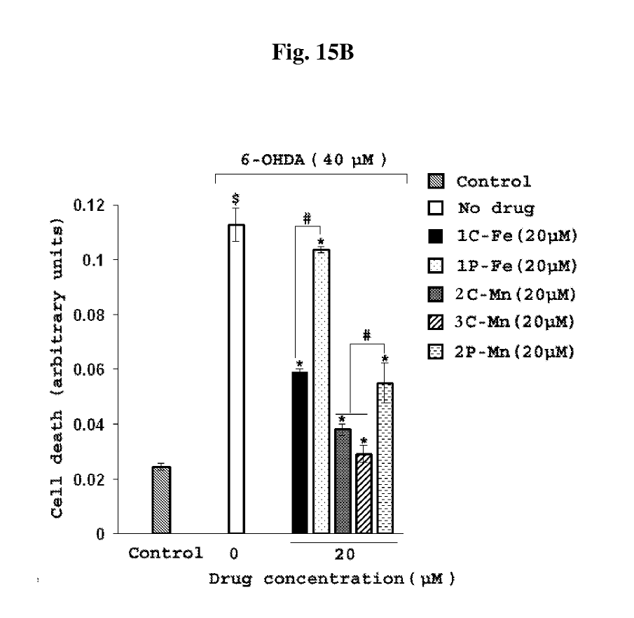

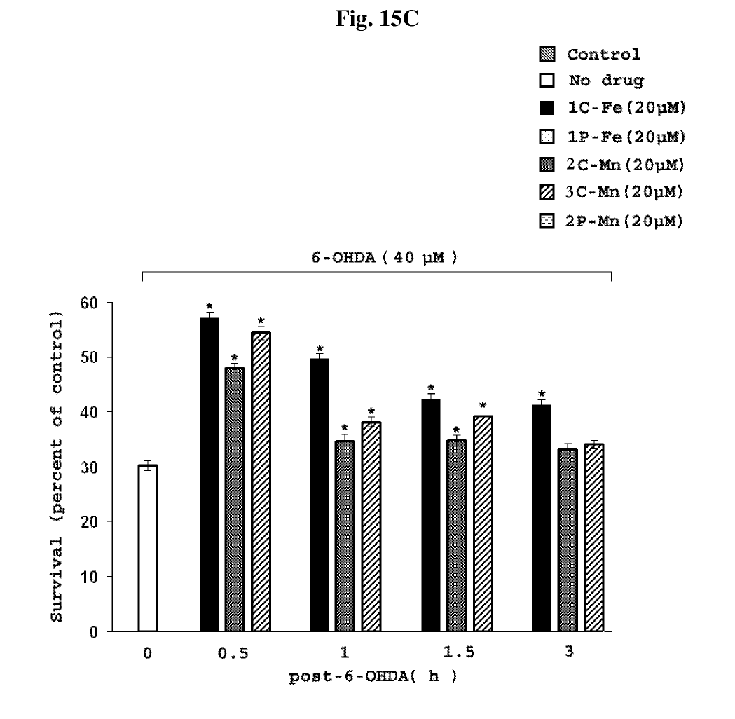

As can be seen in FIGS. 15A-15B, the iron corrole 1C-Fe, as well as the manganese corroles 2C-Mn and 3C-Mn, significantly attenuated 6-OHDA-induced cytotoxicity in SH-SY5Y cell cultures. The magnitude of the neuroprotective effect was more pronounced for the manganese corroles. Additional neurorescue experiments (FIG. 15C) revealed that the iron and manganese corroles confer pronounced neuroprotective effect at 0.5 h post-administration of 6-OHDA, that becomes gradually reduced up to 3 h after exposure to the insult. To further confirm the neuroprotective effect of the corroles in SH-SY5Y cells exposed to 6-OHDA, we employed an immunofluorescence analysis, using a specific primary antibody against the axonal marker, growth-associated protein (GAP-43), and DAPI-staining for nuclear labeling.

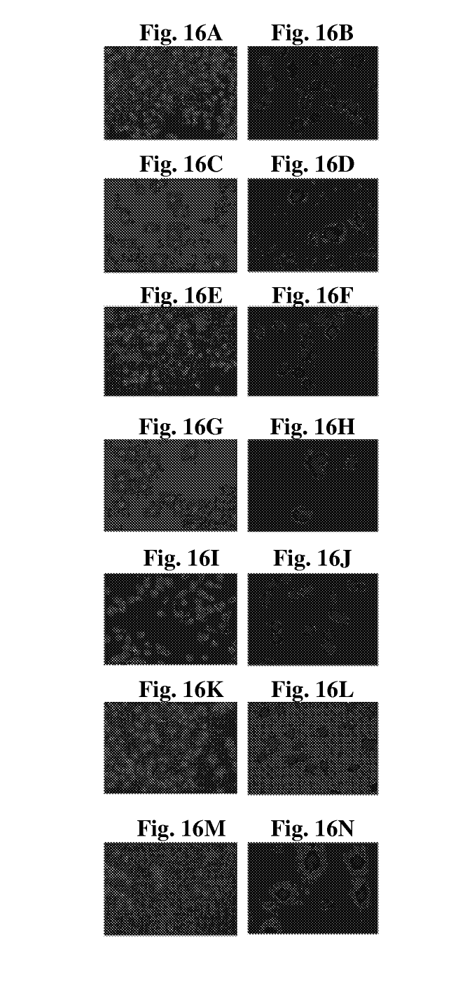

FIGS. 16A-N show that treatment with the corroles 1C-Fe, 2C-Mn and 3C-Mn (20 μM) significantly attenuated 6-OHDA-induced cell mortality, improved cell morphology and reduced the number of apoptotic nuclei, as compared with vehicle-treated cells (16A-D). Furthermore, all three corroles (1C-Fe, 2C-Mn and 3C-Mn) exhibited a remarkable superiority over the porphyrins 1P-Fe and 2P-Mn, in agreement with the results obtained by MTT test and cell death ELISA analysis.

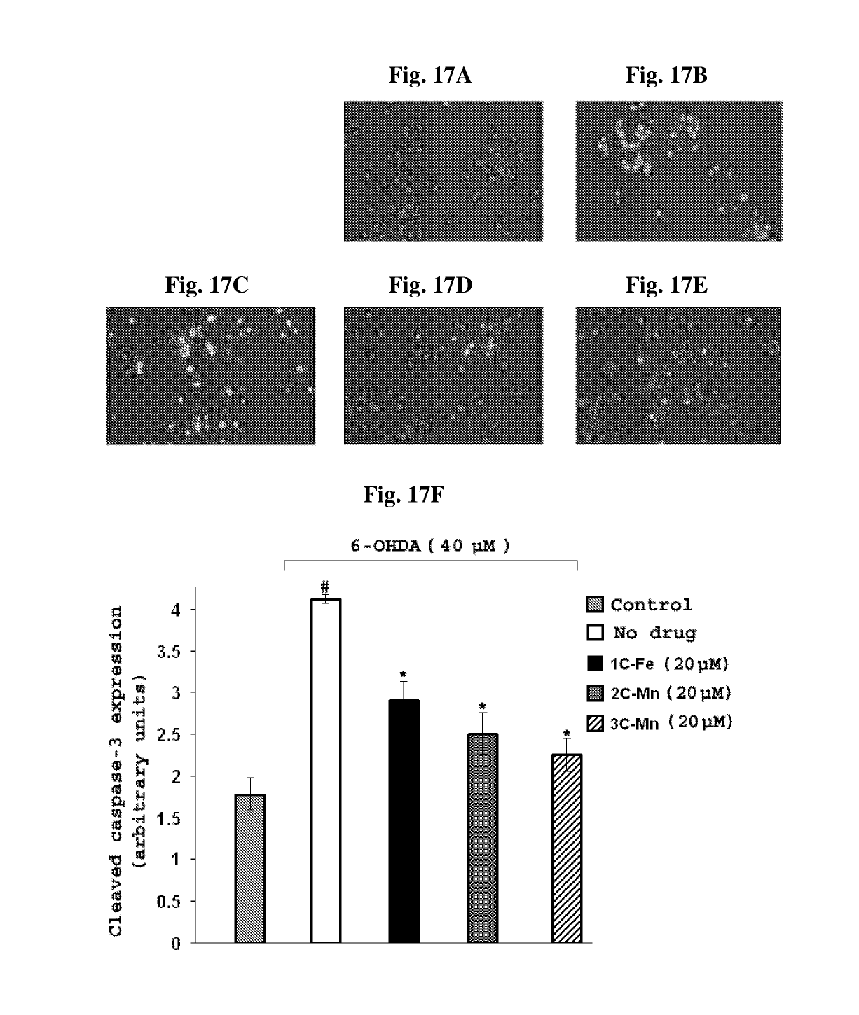

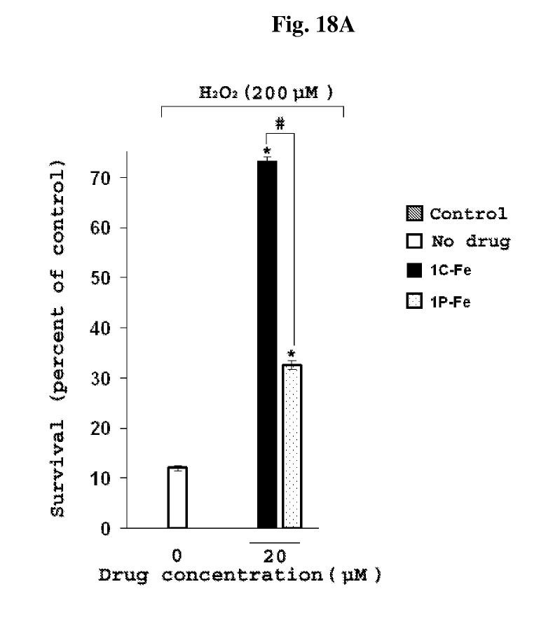

Since 6-OHDA is also known to induce apoptosis in various cell types, including SH-SY5Y (von Coelln et al. 2001; Nie et al. 2002; Shimizu et al. 2002; Jordan et al. 2004), we further tested effect of corroles on the levels of the apoptotic marker, cleaved caspase-3, a major executer of the mitochondrial intrinsic pathway of apoptosis. FIGS. 17A-E show that SH-SY5Y cells incubated with vehicle and exposed to 6-OHDA (40 μM) exhibited a significant increase in the levels of cleaved caspase-3 versus control cells. This effect on cleaved caspase-3 was abolished partially by iron corrole 1C-Fe administration or totally by administration of manganese corroles 2C-Mn or 3C-Mn, as indicated by immunofluorescence analysis (FIG. 17F).

### Example 15

### Comparative Effects of Corroles 1C-Fe, 2C-Mn and Porphyrins 1P-Fe, 2P-Mn Against Toxicity Induced by SIN-1 or by H₂O₂ in NSC-34 Cells

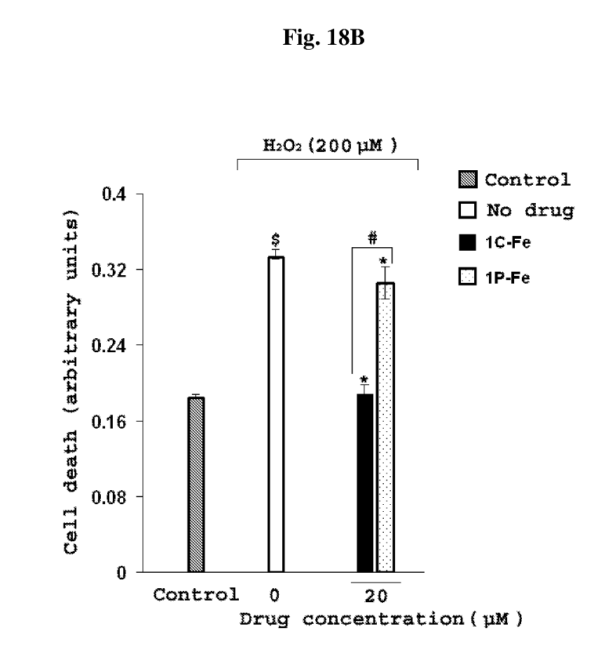

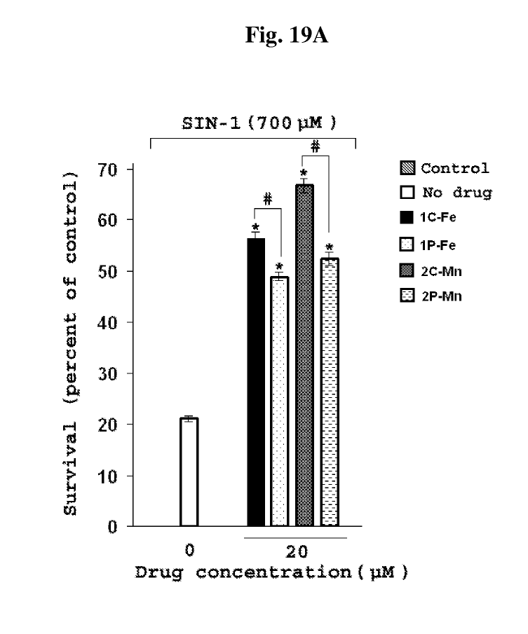

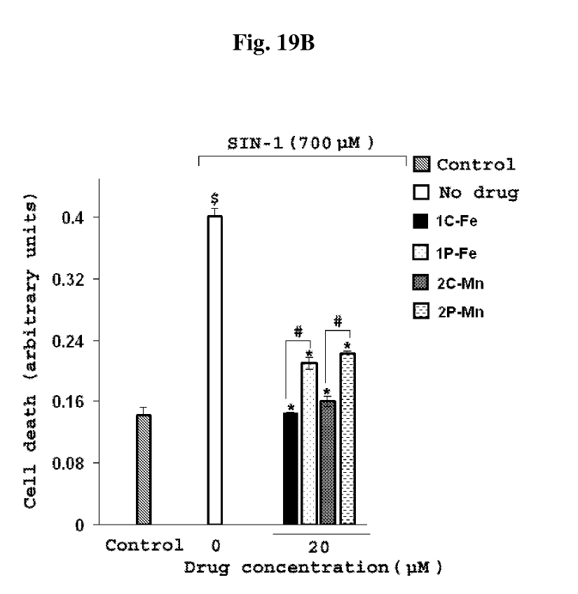

The neuroprotective effects of the iron corrole 1C-Fe and analogous porphyrin 1P-Fe against H₂O₂ insult were further examined in the motor neuron cell line, NSC-34. NSC-34 cell is a hybrid neuroblastoma x spinal cord (NSC) cell line that resembles motor neurons, displaying a multipolar neuron-like phenotype. Mouse motoneuronal NSC-34 cells were pretreated with or without the corroles 1C-Fe or 2C-Mn or the structurally-related porphyrins 1P-Fe, 2P-Mn, respectively, 30 min before exposure to H₂O₂ (200 μM) or SIN-1 (700 μM) for a subsequent 24 h period. Cell viability (FIGS. 18A, 19A) was evaluated by MTT test and cell death (FIGS. 18B, 19B) was assessed using the apoptotic cell death detection ELISA kit. [The graphs in FIGS. 18A-18B and 19A-19B present results expressed as percentage of untreated control. Data are expressed as mean±SEM (n=6) of a representative experiment that was repeated twice with similar results.

As shown in FIGS. 18A-18B for corrole 1C-Fe (20 μM) and porphyrin 1P-Fe, the corrole 1C-Fe was found to confer significant protection against H₂O₂-mediated cytotoxicity in NSC-34 cells, in both MTT (FIG. 18A) and ELISA (FIG. 18B) assays. Consistent with the results in SH-SY5Y cells, it was found that the porphyrin 1P-Fe was not markedly effective in preventing H₂O₂-mediated cytotoxicity in NSC-34 cells. As shown in FIGS. 19A-19B for corroles 1C-Fe and 2C-Mn (20 μM) and porphyrins 1P-Fe and 2P-Mn, both the iron corrole 1C-Fe (20 μM) and the manganese corrole 2C-Mn (20 μM) significantly attenuated SIN-1 (700 μM)-induced cytotoxicity in NSC-34 motor neuron cells, revealing higher cytoprotective activity than their respective analogous porphyrins 1P-Fe and 2P-Mn.

### Example 16

### Neuroprotective Effect of Iron Corroles 1C-Fe and 5C-Fe in Cell Culture Model of Familial ALS

Mutant-superoxide dismutase (G93A-SOD1) is associated with familial amyotrophic lateral sclerosis (FALS). A cell culture model of FALS was implemented by stably transfecting mouse motoneuronal NSC-34 cells or SH-SY5Y cells, to express mutant G93A-SOD1 at levels approximating those seen in the human disease. NSC-34 cells or SH-SY5Y cells were incubated in Dulbecco's modified Eagle's/F-12 medium supplemented with 10% FCS tetracycline-free (FCS Tet-free; Clontech), at 37° C. in an atmosphere of 5% CO2 in air. These cell lines are stably transfected with the pTet-ON plasmid (Clontech, Palo Alto, Calif.) coding for the reverse tetracycline controlled transactivator (rtTA). Induction of mutant SOD1 expression was obtained by shifting nonconfluent cultures into growth medium containing 1% N₂ supplement (Invitrogen, Carlsbad, Calif.) and 1 mg/ml doxocyclin for 48 h in the presence or absence of corrole 1C-Fe and 5C-Fe.

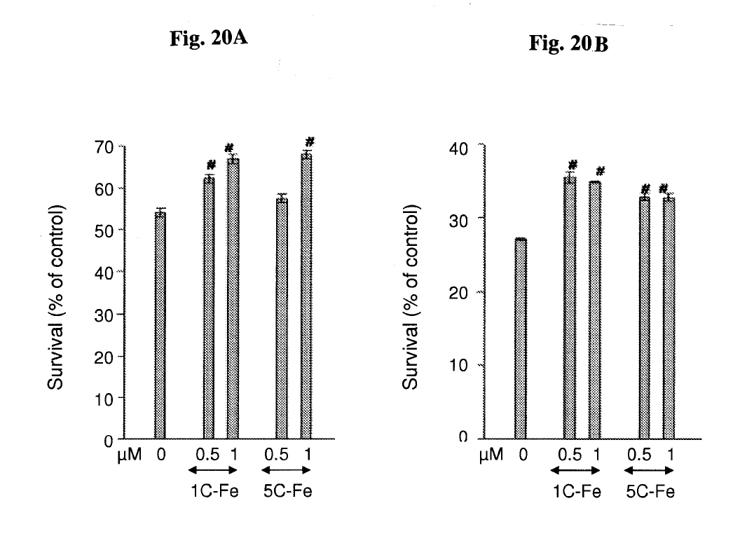

The results are shown in FIGS. 20A-20B. In the induced mutant cells, survival was decreased by more than 40% while treatment with either corrole 1C-Fe or 5C-Fe significantly improved cell viability of NSC-34 G93A cells even at a concentration as low as 0.5 μM (FIG. 20A). Similar results were obtained when the G93A mutation was expressed in SH-SY5Y cells (FIG. 20B).

### Example 17

### Cellular Uptake of the Corroles

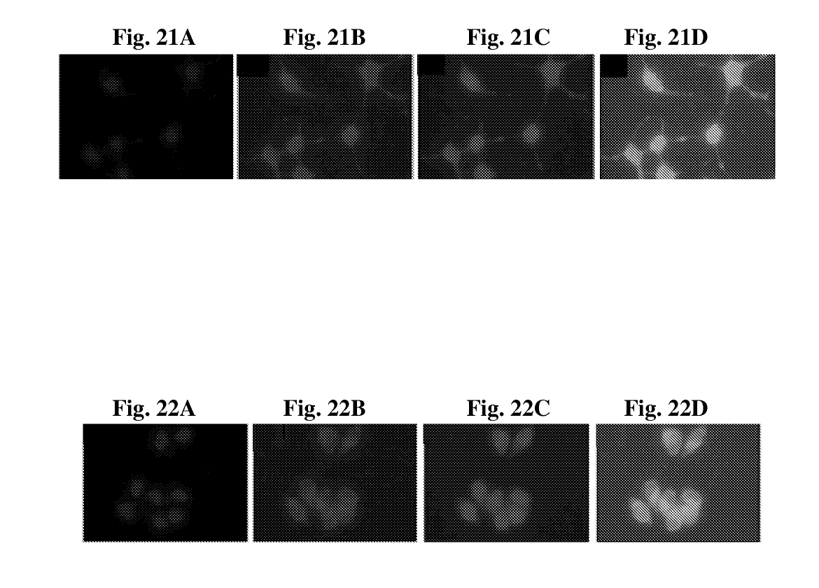

For testing cellular membrane penetration, as well as uptake and accumulation of corroles in the cells, we took advantage of the fluorescence property of an appropriate corrole. For this purpose we used a fluorescent corrole complex (1C-Ga) that is structurally identical to the catalytic compounds utilized in the cytoprotection/cytorescue experiments, except that the chelated metal is gallium(III) rather than iron(III) in 1C-Fe or manganese(III) in 1C-Mn. The results are shown in FIGS. 21A-D for RIN-m (insulinoma) cell line and FIGS. 22A-D for SH-SY5Y (neuroblastoma) cell line. Both figures demonstrate corrole's accumulation inside the cells originated from different cell lines.

To visualize intracellular corrole we used fluorescence microscopy. Neuroblastoma SH-SY5Y cells and insulinoma RIN-m cells were plated on coverslips. At 24 h after plating, corrole 1C-Ga was added directly to the cell media and incubated for half an hour at 37° C. The cells were then aspirated from the media and washed with phosphate buffer to eliminate background signal due to extra cellular corrole. Fixation with formaldehyde was performed. Coverslips with mounting medium (with DAPI) were directly placed on slides and viewed by fluorescence microscopy; the results are shown in FIGS. 21, 22.

From these results we observe that the corrole probably accumulates in the cytoplasm of neuroblastoma and insulinoma cells, but remains excluded from the nucleus. For more accurate and specific analysis of corrole distribution between cellular organelles, specific staining and confocal microscopy will be utilized in the future.

### Example 18

### Preparation of 2,17-bis-sulfonato-5,10,15-tris(p-methoxy-tetra-fluorophenyl)iron(III) corrole (Corrole 6C-Fe, Scheme 1)

### 18.1 Preparation of 5,10,15-tris(p-methoxy-tetrafluorophenyl)corrole

200 mg of 5,10,15-tris(pentafluorophenyl)corrole was dissolved in 100 mL of sodium methoxide solution (0.5 M in methanol). The solution was heated to reflux for 6 hr under argon, followed by evaporation of the solvent. The product was purified by two subsequent silica gel columns (the eluent was ethanol for the first column and CH₂Cl₂/n-hexane 2:1 for the second column), affording 160 mg (77% yield) of 5,10,15-tris(paramethoxytetrafluorophenyl)corrole. ¹H NMR (300 MHz, CDCl₃) δ=9.02 (d, J=4.0 Hz, 2H), 8.73 (d, J=4.8 Hz, 2H), 8.54 (d, J=4.8 Hz, 2H), 8.51 (d, J=4.0 Hz, 2H), 4.31 (s, 9H). ¹⁹F NMR (282.4 MHz, CDCl₃) δ=−139.6 (dd, J¹=22 Hz, J²=7.0 Hz, 2F), −140.1 (dd, J¹=22 Hz, J²=7.0 Hz, 4F), −158.2 (dd, J¹=22 Hz, J²=7.0 Hz, 4F), −158.6 (dd, J¹=22 Hz, J²=7.0 Hz, 2F).

### 18.2 Preparation of 2,17-bis-sulfonato-5,10,15-tris(p-methoxy-tetrafluoro-phenyl)corrole (Corrole 6C, Scheme 1)

100 mg of 5,10,15-tris(p-methoxy-tetrafluorophenyl)corrole and 10 ml of sulfuric acid was stirred at 25 C for 4 hr, after which the reaction mixture was cooled by an ice bath and treated with small ice chips (5-10 g). The acid was neutralized by sodium carbonate, and the product was separated from the sodium sulfate via adding ethanol, filtration and evaporation. The product was purified by silica gel column (the eluent was CH₂Cl₂/ethanol 2:1), affording 80 mg (67% yield) of 2,17-bis-sulfonato-5,10,15-tris(p-methoxytetrafluorophenyl)corrole. ¹H NMR (300 MHz, CD₃OD) δ=9.67 (s, 1H), 8.57 (s, 1H), 8.38 (d, J=4.8 Hz, 1H), 8.22 (d, J=4.5 Hz, 1H), 8.15 (d, J=4.8 Hz, 1H), 8.14 (d, J=4.5 Hz, 2H), 4.24 (s, 3H), 4.23 (s, 3H), 4.21 (s, 3H). ¹⁹F NMR (282.4 MHz, CD₃OD) δ=−140.9 (dd, J¹=24 Hz, J²=8.0 Hz, 2F), −141.9 (dd, J¹=24 Hz, J²=8.0 Hz, 2F), −142.1 (dd, J¹=24 Hz, J²=8.0 Hz, 2F), −161.6 (dd, J¹=24 Hz, J²=8.0 Hz, 2F), −162.1 (dd, J¹=24 Hz, J²=8.0 Hz, 2F), −164.3 (dd, J¹=24 Hz, J²=8.0 Hz, 2F). MS (TOF LD−) m/z (%) 1011.9 (100%) [M²⁻+Na⁺).

### 18.3 Preparation of 2,17-bis-sulfonato-5,10,15-tris(p-methoxytetrafluorophenyl)iron(III) corrole (6C-Fe)

One portion of FeCl₂.4H₂O (100 mg) was added at once to pyridine solution (10 ml) of 2,17-bis-sulfonato-5,10,15-tris(paramethoxytetrafluorophenyl)corrole (100 mg), and the mixture was heated immediately to reflux for 10 min. The product was purified by silica gel column (the eluent was ether/ethanol 3:1 at the beginning then ether/ethanol 1:2), affording 75 mg (71% yield) of the title product 2,17-bis-sulfonato-5,10,15-tris(paramethoxytetrafluorophenyl)iron(III) corrole. ¹⁹F NMR (282.4 MHz, CD₃OD) δ=−109.2 (1F), −119.3 (2F), −153.4 (1F), −154.8 (1F), −157.4 (1F). MS (TOF LD−) m/z (%) 1065.9 (100%) [M²⁻+Na⁺).

### Example 19

### Preparation of 2,17-bis-sulfonato glycine-5,10,15-tris(penta-fluorophenyl)iron(III) corrole (corrole 5C-Fe, Scheme 1)

### 19.1 Preparation of 2,17-bis-sulfonato glycine ethyl ester-5,10,15-tris(pentafluorophenyl) corrole

A solution of 2,17-bis-sulfonylchloride-5,10,15-tris(pentafluoro-phenyl) corrole (70 mg) and 200 μl glycine ethyl ester in CH₂Cl₂ (20 ml) was stirred for 1 hr. The solution was washed twice with a solution of HCl (2 M) and then with distilled water. The solvent was evaporated and 2,17-bis-sulfonato glycineethylester-5,10,15-tris(pentafluorophenyl) corrole was obtained in quantitative yield. ¹⁹F NMR (188 MHz, CDCl₃) δ=−136.8 (broad peak, 4F), −139.0 (broad peak, 2F), −150.7 (broad peak, 1F), −151.1 (broad peak, 2F), −160.7 (broad peak, 4F), −162.5 (broad peak, 2F), MS (TOF LD+) m/z (%) 1149.2 (100%) [M+Na⁺).

### 19.2 Preparation of 2,17-bis-sulfonato glycine ethyl ester-5,10,15-tris(pentafluorophenyl)iron(III) corrole

One portion of FeCl₂.4H₂O (70 mg) was added at once to pyridine solution (10 ml) of 2,17-bis-sulfonato glycineethylester-5,10,15-tris(pentafluorophenyl) corrole (70 mg), and the mixture was heated immediately to reflux for 10 min. The product was purified by silica gel column (the eluent was CH₂Cl₂/n-hexane 1:1 at the beginning then CH₂Cl₂/THF 100:1), affording 60 mg (82% yield) of 2,17-bis-sulfonato glycine ethyl ester-5,10,15-tris(pentafluoro-phenyl)iron(III) corrole. ¹⁹F NMR (282.4 MHz, CDCl₃) δ=−118.1 (2F), −123.0 (2F), −124.9 (2F), −153.7 (2F), −156.1 (1F), −160.9 (2F), −161.2 (2F), −163.2 (2F). MS (TOF LD−) m/z (%) 1179.242 (80%) [M), 1202.244 (100%) [M+Na]).

### 19.3 Preparation of 2,17-bis-sulfonato glycine-5,10,15-tris(pentafluoro-phenyl)iron(III) corrole

60 mg of 2,17-bis-sulfonato glycine ethyl ester-5,10,15-tris(pentafluoro-phenyl)iron(III) corrole and 100 ml of water that contain 500 mg sodium carbonate was refluxed for 1 hr, after which the basic solution was neutralized by HCl and washed with CH₂Cl₂. The product transferred to the organic phase. The CH₂Cl₂ washed twice with water and dried by sodium sulfate. 45 mg of the end product 2,17-bis-sulfonato glycine-5,10,15-tris(pentafluorophenyl)iron(III) corrole were obtained (79% yield) after filtration and evaporation of the CH₂Cl₂ solution. ¹⁹F NMR (282.4 MHz, CDCl₃) δ=−118.2 (2F), −123.1 (2F), −125.0 (2F), −153.8 (2F), −156.1 (1F), −161.1 (4F), −163.3 (2F). MS (TOF LD−) m/z (%) 1122.468 (100%) [M]).

### Example 20

### Preparation of Corroles 3C-Mn and 4C-Mn (Scheme 1)

The preparation of the metal-free and nonalkylated corroles 3C and 4C, from which the metal complexes 3C-Mn and 4C-Mn, respectively, were prepared, is described in Saltsman et al., 2008.

### 20.1 Preparation of 5,10,15-tris(o-pyridyl)corrole (3C, the precursor of 3C-M, Scheme 1)

For the preparation of title compound in Example 20.1, 2-pyridinium dipyrromethane and appropriate aldehyde (2-pyridine carboxaldehyde/pentafluorobenzaldehyde) were reacted as described by Gryko and Piechota, 2002. Shortly, samples of dipyrromethane (0.4 mmol) and the appropriate aldehyde (0.2 mmol) were dissolved in CH₂Cl₂ (12 mL) and trifluoroacetic acid (62 μL, 0.8 mmol) was added to this stirring mixture at room temperature. After 1 h, triethylamine (112 μL, 0.8 mmol) was added and the reaction mixture was diluted with CH₂Cl₂ (308 mL) DDQ (90 mg, 0.4 mmol) was added and stirring was continued for a further 10 min. The reaction mixture was evaporated to dryness and purified by column chromatography on silica, with the purification details that are described for each case.

The reaction mixture was purified via column chromatography on silica. The second blue-green band was eluted with ethyl acetate: n-hexane (at first, 3:1 and then gradually up to 100% ethyl acetate, then 10% methanol in ethyl acetate). Second chromatographic treatment (ethyl acetate: n-hexane, 3:1, then 3% n-hexane in ethyl acetate, then 5% methanol in ethyl acetate) provided pure corrole (25 mg, 24% yield), R_{f} (silica, ethyl acetate)=0.24 ¹H 500 MHz NMR(C₆D₆) δ=8.81 (br s, 2H), 8.79 (d, ³J (H,H)=4.12 Hz, 2H), 8.67 (br s 1H), 8.65 (d, ³J (H,H)=4.35 Hz, 2H), 8.44 (d, ³J (H,H)=4.12 Hz, 2H), 8.23 (d, ³J (4.58 Hz, 2H), 8.04 (d, ³J (H,H)=7.56 Hz, 2H), 7.91 (d, ³J (H,H)=7.33 Hz, 1H), 7.34 (m, 3H), 6.92 (m, 3H). UV-vis (ethylacetate): λₘₐₓ, nm (ε×10⁻³) 418 (36.3), 582 (6.7), 614 (4.4).

MS (MALDI-TOF): m/z (%): 528.3 [M⁻, 100%]; 530.5 [M⁺, 100%].

### 20.2 Preparation of 10-(pentafluorophenyl)-5,15-bis(o-pyridyl)corrole (4C)

The reaction mixture from the reaction with pentafluorobenzadehyde was purified via column chromatography on silica. The second green-blue band that was eluted with ethyl acetate: n-hexane (1:4, then 1:2) provided a fraction that contained corrole 4C. Final purification of 4C was achieved by preparative thin-layer chromatography (silica plate, ethyl acetate: n-hexane, 3:4) as to afford pure 4C (27 mg, 22% yield), R_{f} (silica, ethyl acetate: n-hexane, 2:3)=0.81. ¹H 300 MHz NMR(C₆D₆): δ=8.74 (br s, 2H), 8.61 (d, ³J (H,H)=4.12 Hz, 2H), 8.22 (d, ³J (H,H)=4.67 Hz, 2H), 8.09 (d, ³J (H,H)=7.96 Hz, 2H), 7.71 (d, ³J (H,H)=4.94 Hz, 2H), 7.06 (m, 4H), 6.42 (m, 2H), −1.47 (br s, 3H). ¹⁹F 282 MHz (C₆D₆): δ=138.36 (dd, ³J (F,F)=25.3 Hz, ⁴J (F,F)=5.6 Hz, 2F), −154.16 (t, ³J (F,F)=22.6 Hz, 1F), −162.96 (td, ³J (F,F)=25.4 Hz, ⁴J (F,F)=8.5 Hz, 2F). UV-vis (EtOAc): λₘₐₓ, nm (ε×10⁻³) 416 (48.5), 578 (10.3).

MS (MALDI-TOF): m/z (%): 617.0 [M⁻, 100%]; 619.2 [M⁺, 100%].

MS (MALDI-TOF): m/z (%): 619.2 [M⁺, 100%].

### 20.3 Preparation of N-methylated 3C and 4C

3C and 4C were dissolved in the minimum volume of DMF and excess methyl iodide (100 eqv) was added to the solutions. Reaction mixtures were stirred at room temperature overnight. Small amount of methanol and three-fold excess of diethyl ether were added the reaction mixtures. Precipitated products were collected and washed with addition portion of diethyl ether.

5,10,15-tris(N-methyl-o-pyridiniumyl)corrole (3C³⁺): was obtained as a mixture of three atropoisomeric structures, which were separated by reversed-phase HPLC. R_{f} (silica, KNO₃ sat:H₂O:acetonitrile, 1:1:8)=0.46. UV-vis (acetonitrile): λ_{λmax}, nm (ε×10⁻³) 408 (8.0), 436 (8.2), 626 (3.3).

MS (MALDI-TOF) ES⁺ (CH₃CN): m/z (%): 575 (10) [M⁺], 560 (40) [M−15(CH₃)], 545 (100) [M−30(2xCH₃)], 287 (100) [M⁺]/2, 196 (25) [M+16(O)]/3.

10-(pentafluorophenyl)-5,15-bis(N-methyl-o-pyridyniuml)corrole (4C²⁺): was obtained as a mixture of two atropoisomers, which were separated by reversed-phase HPLC. R_{f} (silica, KNO₃ sat:H₂O:acetonitrile, 1:1:8)=0.55. ¹H 300 MHz NMR CD₃CN): δ=9.15 (d, ³J (H,H)=4.16 Hz, 2H), 9.12 (d, ³J (H,H)=6.46 Hz, 2H), 8.69 (m, 4H), 8.56 (d, ³J (H,H)=4.74 Hz, 2H), 8.52 (d, ³J (H,H)=4.22 Hz, 2H), 8.49 (d, ³J (H,H)=4.67 Hz, 2H), 8.31 (t, ³J (H,H)=6.59 Hz, 2H), 4.15 (s, 3H), 4.13 (s, 3H). ¹⁹F 282 MHz (CD₃CN): δ=−141.01 (m, 2F), −158.21 (t, ³J (F,F)=19.4 Hz, 1F), −165.01 (m, 2F). UV-vis (acetonitrile): λₘₐₓ, nm (ε×10⁻³)

MS (MALDI-TOF) ES⁺ (CH₃CN): m/z (%): 648.2 (100) [M⁺], 633.2 (85) [M−15(CH₃)], 324.1 (100) [M⁺]/2.

### 20.3 Preparation of Mn(III) complex of 5,10,15-tris(N-methyl-o-pyridynium)corrole (3C-Mn); Mn(III) complex of 10-(pentafluorophenyl)-5,15-bis(N-methyl-o-pyridynium)corrole (4C-Mn)

The title compounds were prepared by heating DMF solution of the methylated 3C³⁺ or 4C²⁺ to reflux with 15 eqv of Mn(OAc)₂.4H₂O. UV-vis and TLC examinations revealed no starting material. Solvent evaporation and carefully separation by chromatographic column of the residue (silica gel, KNO₃sat: H₂O: acetonitrile, 1:1:8 for 3C-Mn and KCl sat: H₂O: acetonitrile, 1:1:8) for 4C-Mn) afforded 73% and 85% yield, respectively, as dark-green solids. 3C-Mn: R_{f} (silica, KNO₃sat: H₂O: acetonitrile, 1:1:8)=0.24. MS (MALDI-TOF) LD⁺ (CH₃CN): m/z (%): 626.1 (10) [M⁺], 611.1 (30) [M⁺−CH₃], 596.1 (100) [M⁺−(2xCH₃)], 581.1 (95) [M⁺−(3xCH₃)]; ES⁺: 279.0 (100)[M⁺/3+(2xCl⁻)].

4C-Mn: R_{f} (silica, KNO₃sat: H₂O: acetonitrile, 1:1:8)=0.78. ¹⁹F 282 MHz (CD₃CN): δ=−119.29 (br s, 2F), −154.06 (m, 1F), −157.51 (m, 2F). MS (MALDI-TOF) ES⁺ (CH₃CN): m/z (%): 350.1 (100) [M⁺]/2.

### Example 21

### Synthesis of 5,15-bis(4-N-propargyl-pyridilium)-10-pentafluoro-phenyl corrolato manganese(III) (E-pr-Mn, Scheme 2)

Corrole bearing pyridine substituents was synthesized as follows: Pentafluorobenzaldehyde (50 μL, 0.4 mmol) was added to a 10 mL solution of 4-pyridyl-dipyrromethane (178 mg, 0.8 mmol) in propionic acid and the mixture was heated to reflux for 50 min. The residue obtained after solvent evaporation was washed with hot water, neutralized with ammonium hydroxide (25%), and washed again with hot water. The solid material was dissolved in methanol, basic alumina was added, and the solvent was evaporated. Separation between corrole and the analogous porphyrin was achieved by column chromatography (silica, CH₂Cl₂ followed by 0.5 methanol) followed by separation by preparative thin-layer chromatography (silica plate, CHCl₃/MeOH 50:1) affording pure corrole (18 mg, 8%).

R_{f}=0.15 (CH₂Cl₂/ethyl acetate 1:1). UV/Vis (CH₂Cl₂/MeOH (2:1)): λₘₐₓ (ε10⁻³)=416 (104.99), 576 (16.14), 610 (9.40), 640 (5.34), MS (MALDI-TOF): m/z (%): 619 (100) [M^{+].} ¹H NMR (200 MHz, C₆D₆): δ=8.93 (br s, 4H). 8.67 (d, J=4 Hz, 4H), 8.26 (m, 4H), 7.90 ppm (br. s, 4H). ¹⁹F (188 MHz): δ=438.79 (d, J=23.5 Hz, 2F), −153.22 (t, J=21.9 Hz, 1F), −162.37 ppm 4, J=225 Hz, 1F).

The corresponding manganese complex was prepared by heating the corrole solution in pyridine at reflux with 15 equivalents of Mn(OAc)₂.4 H₂O followed by chromatographic separation (silica, starting with CH₂Cl₂, and gradually adding methanol), affording 81% yield. UV/Vis (MeOH): λₘₐₓ (ε10⁻³)=368 (16.7), 402 (26.7), 420 (4.8), 458 (18.4), 484 (16.2), 634 (9.4). MS (MALDI-TOF LD⁺): m/z (%): 670 (100) [M⁺]. ¹⁹F (C₅D₄N) (1.88 MHz): δ=−136.58 (br s, 2F), −155.16 (s, 1F), −161.23 ppm (s, 2F)

N-alkynylation: The manganese complex was dissolved in hot THF and excess propargyl bromide was added to the solution, which were then left at 50° C. until complete precipitation. The solid material was collected by centrifugation and washed with THF and diethyl ether until the solvent was colorless, UV/Vis (methanol): λₘₐₓ 462, 600, 638; MS (MALDI-TOF LD⁺): m/z (%): 711 (10) [M⁺], 671 (80) [M−80]. ¹⁹F (MeOD) (188 MHz): δ=−132.1 (br s, 2F), −144.71 (s, 1F), −161.70 ppm (s, 2F), The tide product was crystallized by diffusion of diethyl ether into concentrated methanol solution, affording 58% yield.

### Example 22

### Neuroprotection by Corroles In Vivo in the MPTP Model of PD

The mouse model of Parkinson's disease (PD) using the neurotoxin 1-methyl-4-phenyl-1,2,3,6-tetrahydropyridine (MPTP) recapitulates many of the pathological manifestations encountered in human PD including increased generation of ROS and nitration of tyrosine residues on proteins in the substantia nigra (SN) of the ventral midbrain, a region containing the vulnerable DA neurons. Employing this model it was recently shown that rasagiline and M-30 (a propargyl-containing iron chelator disclosed in WO 2004/041151) are neuroprotective and also have the ability to restore nigral dopaminergic neurons when given post-MPTP (Gal et al., 2005). Based on our preliminary results (see above, FIGS. 3-20) and on the somewhat larger lipophylicity of corrole 5C-Fe (higher probability to penetrate the BBB), this was the first corrole tested. Mice are treated with corrole 5C-Fe (1, 2, 10 and 20 mg/kg, once a day, by oral or intraperitoneal (i.p.) administration) for 5 consecutive days and followed by the combination with MPTP (24 mg/kg, i.p.) for 4 additional days. Monoamine oxidase (MAO) A/B activity is assessed in the striatum and hippocampus, as well as tyrosine hydroxylase (TH) activity and catecholamine levels in the striatum, hypothalamus and dorsal raphe. In addition, parkinsonism-associated proteins, such as α-synuclein, and synphilin-1, and iron regulatory proteins, such as ferritin, transferrin receptor, and hypoxia inducible factor (HIF)-1 are monitored for changes in striatal expression. To link the possible neuroprotective effects of the corrole with antioxidant actions, parameters of ROS and RNS including reduced glutathione (GSH), lipid peroxidation and 3-nitrosylated proteins are tested in the mouse ventral midbrain region, as described (Liang et al., 2007).

### Example 23

### Neurorescue by Corroles in the MPTP Model

Our preliminary results have shown that corroles 5C-Fe and 1C-Fe possess not only preventive but also potent neurorescue activity (indicated by their post-damage administration) in cell cultures (see FIGS. 8, 11, 14 and 15). Therefore, corrole 5C-Fe is the one initially investigated for its potential neurorescue/neurorestorative effect in post-MPTP-induced nigrostriatal dopamine neurodegeneration model of PD in mice. MPTP (20 mg/kg, i.p, per day) administration for 4 days is followed by a further 4 days resting period (day 8) to allow for the full conversion of MPTP to its active metabolite, MPP+ (Sagi et al., 2007). At day 8, corrole 5C-Fe (doses are according to the neuroprotective paradigm results) is administered (i.p. or orally) for 14 days. The same parameters described for the neuroprotective studies in Example 22 are determined here as well.

### Example 24

### Molecular Effects of Corroles In Vitro on Apoptosis, Cell Signaling, and OS Related Processes

The molecular mechanism of cyotoprotective/cytorescue action of the various corroles is assessed in the following cell lines: clonal mouse-derived SN dopaminergic neuronal cell line, SN4741 (PD model), mouse motor neuronal cell line, NSC-34 (ALS model) and in rat insulinoma INS-1E and RIN-m cell lines (DM model). Apoptosis-associated protein markers (e.g. the Bcl-2 family proteins Bax, Bad, Bcl-2; cytochrome c; phospho-H2AX; cleaved caspase-3 and -8; cleaved poly ADP-ribose (PARP)), signaling cascades (e.g. MAPK, PKC, PI3K/AKT) and OS parameters (catalase, GSH, glutathione peroxidase, endogenous ROS levels), are determined as described in earlier publications of the M. Youdim group (Weinreb et al., 2004; Bar-Am et al., 2005). Specifically for the insulinoma cell lines, the effect of the corroles against alloxan, a diabetogenic toxin which selectively kills the insulin-producing beta-cells of the pancreas, are investigated and both cellular and secreted insulin levels, are determined. Also, the levels of thioredoxin-interacting protein, a pro-apoptotic factor recently found to be increased in beta cell subjected to glucose toxicity, is measured (Chen et al., 2006).

### Example 25

### Effect of Corroles on Mitochondrial Function

Various corroles are examined with respect to their ability to prevent the collapsing of the mitochondria and consequent decrease in proteasomal activity (because of impairment in ATP supply) following exposure to 6-OHDA, a neurotoxin known to inhibit mitochondrial complexes I and IV. Mitochondrial membrane potential (ΔΨm) is determined by the JC-1 test. Briefly, the corroles are added 30 min before exposure to 6-OHDA (35 μM), for a period of 4 h. Changes in ΔΨm are determined by JC-1 mitochondrial membrane potential detection kit (Biotium, Inc) for 20 min at 37° C. Dual emission images (530 and 590 nm) represent the signal from monomeric (green) and J-aggregate (red) JC-1 fluorescence by confocal microscopy. At high-membrane potentials characteristic of energized mitochondria, JC-1 accumulates sufficiently to aggregate, resulting in large, orange (590 nm) shifts in the emission maximum. At lower potentials, the dye exists as a green fluorescent (530 nm) monomer).

### Example 26

### Biodistribution and Bioavailability of Corroles

Issues regarding biodistribution, transport mechanisms, accumulation in specific organs, and the eventual elimination of administered compounds are among the prime concerns when it comes to real life utilization of potential drugs. This can be addressed by several and quite different means, including in vitro, in vivo and ex-vivo examinations. One way to predict distribution is via examination of the binding of the corroles to circulating proteins that might act as transporters, similar to experiment performed previously by the inventors with metal complexes of corrole 1C (Scheme 1), which spontaneously form tightly bound non-covalent bioconjugates with HDL (Haber et al., 2007), albumin (Mahammed et al., 2004), and transferrin (Haber and Gross. 2007) (listed in increasing order of affinity), as well as with semi-synthetic proteins (Agadjanian et al., 2006). Preliminary experiments revealed that the metal complexes of corrole 2C (such as 2C-Mn) do not bind to albumin, but still do so for transferrin, one of the very few proteins that may carry drugs to the brain. A more direct approach relies on the strong fluorescence of corroles that are not chelated by transition metals (no metal such as in corroles 1C and 2C, or using corrole Ga/Al complexes such as 1C-Ga). This feature is used for testing cell entry via confocal microscopy (Agadjanian et al., 2006), without the need of attaching a fluorescent tag. Furthermore, it may even be used for whole animal imaging purposes, as shown in Example 4 hereinabove. Positron Emission Tomography (PET) may also be used relying on copper corroles such as corrole 1C-Cu. The half-life of ⁶⁴Cu is 12.7 hours, copper may be inserted into corroles within minutes at room temperature and the metal does not leach from the corrole once inserted.

Tissue distribution of the corroles is determined in various brain areas including the cortex, hippocampus and brain stem, as well as their levels in the periphery and in plasma at different time intervals (1-96 h), by means of HPLC and chemiluminescence. Importantly, we have already confirmed that nanomolar concentrations of complex 1C-Fe can be determined in plasma samples via the luminol/H₂O₂ method (Motsenbocker et al., 1999).

### Example 27

### The Capacity of Corroles to Prevent Development of Streptozotocin (STZ)-Induced Diabetes in Mice

Following the initial studies using the compounds for in-vitro protection of insulin-producing cells exposed to hydrogen peroxide or SIN-1 (converts into PN at physiological pH), the selected corroles are administered i.p. to mice (the doses are determined from the MPTP model results) for different time periods (1, 3 and 6 weeks) before, during and one week after administration of the diabetogenic-inducing toxin, STZ, which selectively kills the insulin-producing beta-cells in the pancreas. The following experimental groups are studied.

Diabetes Induction and Side Effects Monitoring

Diabetes in Balb/c mice (Harlan Laboratories, Jerusalem) is induced by a single injection of STZ solution (Sigma, St. Louis, Mo.) at a dose of 200 mg/kg body mass or by low multiple doses injections (40 mg/kg×5 days). STZ is injected i.p. after dissolving in citric buffer (pH 4.5). Diabetes severity is estimated by blood and urine glucose monitoring, body weight, as well as by the glucose tolerance test. All animals treated with the corroles are occasionally monitored for possible side effects by blood sampling for biochemical, haematological profiles. These studies include two different groups: mice with high dose STZ diabetes and mice with multiple low doses STZ diabetes, for examining the effects of the corroles on direct toxic beta cell destruction and on autoimmune-like beta cell destruction, respectively.

### Example 28

### Potential Therapeutic Utility of Corroles in APP/Presenilin-1 (PS1), Double Transgenic Mice Model of Alzheimer's Disease (AD)

To address the possible neuroprotective role of the corroles on the pathogenesis of AD, their effects are examined on the regulation/processing mechanisms of amyloid precursor protein (APP) (e.g. APP mRNA/protein, soluble α/βAPP, fibrillar Aβ peptides/plaque deposits) in the cerebral cortex and hippocampus of the doubly transgenic AD, APP/PS1 mice. Daily gavage dosage is given for 4 consecutive weeks before symptoms appearance (˜6 months of age). After drug administration is completed, 5-6 animals from each group are sacrificed; their brains are dissected and stored at −70° C. for further analysis.

### Example 29

### Potential Therapeutic Utility of Corroles in ALS Transgenic Mice

Hemizygous SOD transgenic mice (carrying a high copy number of a mutant allele human SOD1 containing the Gly93-->Ala (G93A) substitution) become paralyzed in one or more limbs at late disease stage, with a lifespan of ˜130 days. Selected corroles are administered by daily gavage beginning at an asymptomatic state (day 70) until death. Motor function, lifespan and post-mortem histopathological analysis are assessed.

### Example 30

### Bifunctional Propargyl-Corroles: MAO-A/B Inhibition and Anti-Depressant Potential

Several hybrid N-propargyl-corroles (Scheme 2) are initially examined in-vitro for their properties as MAO inhibitors. The compounds exhibiting highest potencies are selected for further investigation. Mice are administered with the propargyl-corroles (1-10 mg/kg, i.p.) and sacrificed 1 h later to assess ex-vivo inhibition of MAO A/B activity in various brain regions and systemic organs. In addition to evaluation of MAO inhibitory potency, these findings provide an indication regarding brain permeability of the examined drugs. Assessment of the levels of striatal amines (DA, DOPAC, HVA, NE, 5-HT, and 5-HIAA) are performed as well, to corroborate findings from the MAO inhibition assay (Gal et al., 2005). Propargyl-corroles displaying ability to inhibit MAO-A in the brain are examined for their potency in ameliorating depression. This is performed using the forced swim test model, a specific model of behavioral. despair and depression, as previously described (Weinstock et al., 2002). Rats are administered propargyl-corroles (5 or 10 mg/kg) and assessed for behavior characteristics typical of depression.

Non-selective MAO inhibitors combined with diet-derived tyramine ingestion may lead to what is commonly termed “the cheese reaction”, characterized by life-threatening hypertension, resulting from elevated synapse norepinephrin levels (Youdim and Weinstock, 2004). Due to this side effect, the usage of MAO inhibitors has been limited. Since the predominant MAO isoenzyme in the small intestine is MAO-A, the development of novel MAO-inhibitors has hitherto focused on selective MAO-B inhibition. The propargyl-corroles are examined whether they produce or not such hypertensive effect. This assumption is based on our findings showing that the neuroprotective iron chelator-propargyl containing, M-30, potently inhibits both MAO-A and -B in the brain, but poorly in peripheral organs (Gal et al., 2005). To this end, rats are administered propargyl-corroles (5 and 10 mg/kg, i.p.), prior to oral tyramine administration (Weinstock et al., 2002), and then blood pressure and heart rate is determined. MAO-A and MAO-B inhibition in the brain, liver and intestine is determined as well.

Finally, based on the positive outcome of the above studies, the selected bifunctional corroles are tested for their cyto/neuroprotective properties in the various disease models.

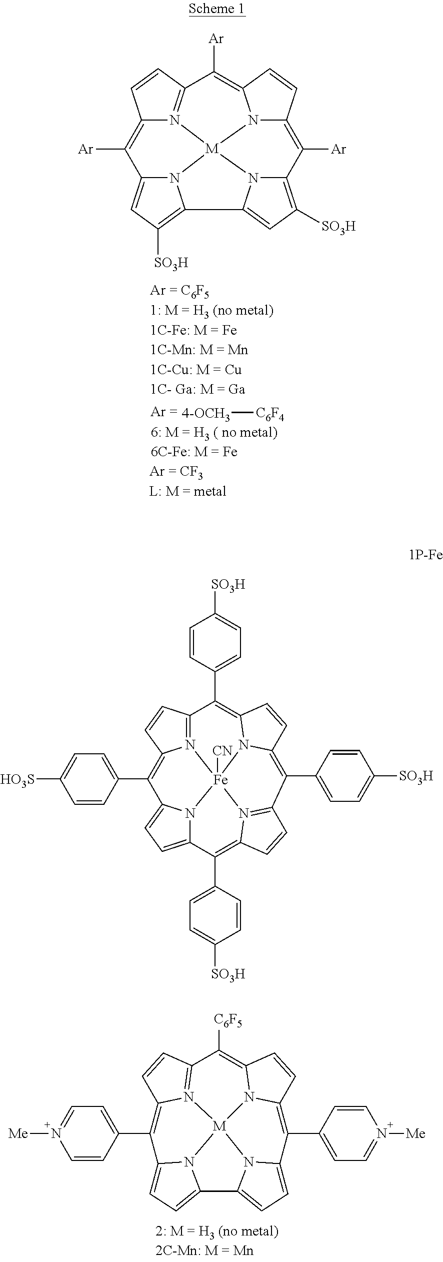

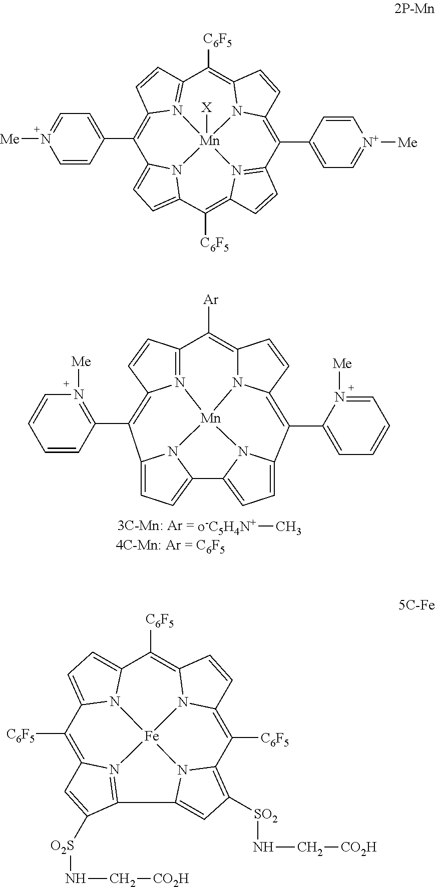

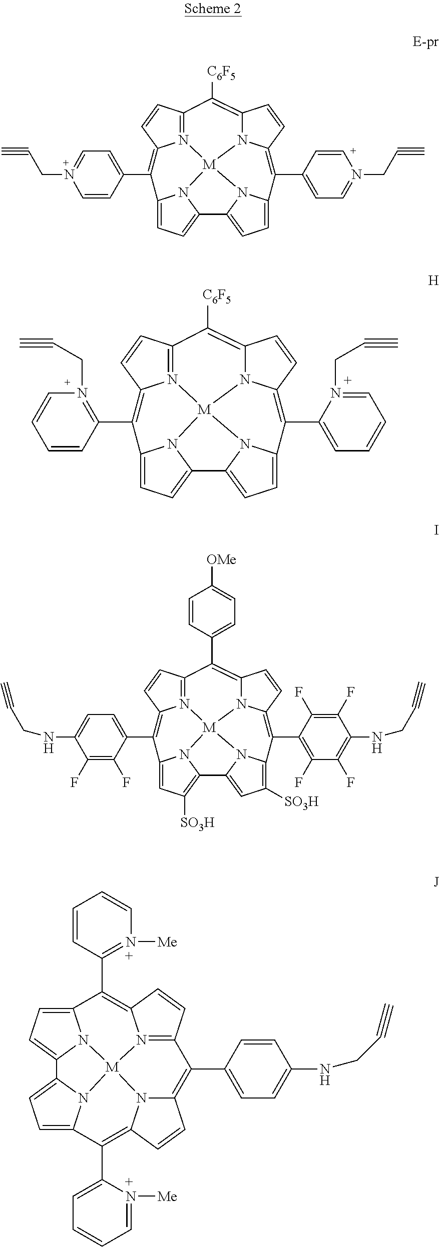

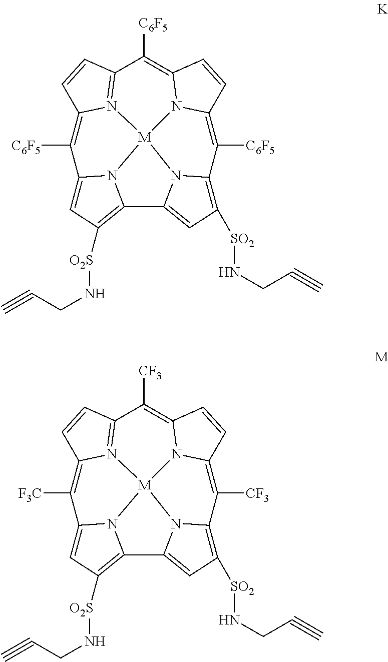

### REFERENCES

Agadjanian H, Weaver J J, Mahammed A, Rentsendorj A, Bass S, Kim J, Dmochowski I J, Margalit R, Gray H B, Gross Z and L. K. Medina-Kauwe. (2006). Specific Delivery of Corroles to Cells via Noncovalent Conjugates with Viral Proteins. Pharmaceutical Res. 23:367-377.
Aviv I, Gross Z. (2007). Corrole-based applications. Chem Commun (Camb).(20): 1987-99.
Avramovich-Tirosh Y, Amit T, Bar-Am O, Zheng H, Fridkin M, Youdim MB (2007). Therapeutic targets and potential of the novel brain-permeable multifunctional iron chelator monoamine oxidase inhibitor drug, M-30, for the treatment of Alzheimer's disease. J. Neurochem. 100: 490-502.
Bar-Am O, Weinreb O, Amit T and Youdim M B (2005). Regulation of Bcl-2 family proteins, neurotrophic factors, and APP processing in the neurorescue activity of propargylamine. FASEB J. 19:1899-901.
Barbe, J. M., Canard, G., Brandes, S. & Guilrad, R (2005). Synthesis and physicochemical characterization of meso-functionalized corroles: Precursors of organic-inorganic hybrid materials. Eur J Org. Chem. 21:4601-4611.
Barnham K J, Masters C L & Bush A I (2004). Neurodegenerative diseases and oxidative stress. Nat Rev Drug Discov. 3:205-214
Batini'c-Haberle I, Spasojevi'c I, Stevens R D, Bondurant B, Okado-Matsumoto A, Fridovich I, Vujas\check{}kovic Z. and Dewhirst M W (2006). New PEG-ylated Mn(III) porphyrins approaching catalytic activity of SOD enzyme. Dalton Trans. 617-624.
Chen J, Couto F M, Minn A H, Shalev A (2006). Exenatide inhibits beta-cell apoptosis by decreasing thioredoxin-interacting protein. Biochem. Biophys. Res. Commun 346:1067-74.
Fisher A E O, Hague T A, Clarke C L and Naughton D P. (2004). Catalytic superoxide scavenging by metal complexes of the calcium chelator EGTA and contrast agent EHPG. Biochem. Biophys. Res. Commun. 323:163-167.
Gal S, Zheng H, Fridkin M, Youdim MB (2005). Novel multifunctional neuroprotective iron chelator-monoamine oxidase inhibitor drugs for neurodegenerative diseases. In vivo selective brain monoamine oxidase inhibition and prevention of MPTP-induced striatal dopamine depletion. J. Neurochem. 95:79-88.
Gershman, Z., Goldberg, I. & Gross, Z (2007). DNA binding and catalytic properties of positively-charged corroles. Angew. Chem. Int Ed Engl 46:4320-4324.
Goldschmidt R, Goldberg I, Balazs Y and Gross Z (2006). Synthesis of a Corrole with Small and Electron-withdrawing Substitutents, 5,15-bistrifluoromethyl-10-pentafluorophenylcorrole. J Porphyrins Phthalocyanines 10:76-86.
Goldstein S., Lind J. & Merenyi G (2005). Chemistry of Peroxynitrites as Compared to Peroxynitrates. Chem. Rev 105:2457-2470. the mouse, Biochem J. 199: 393-398.
Gross Z, Galili N, and Saltsman I (1999). The First Direct Synthesis of Corroles from Pyrrole, Angew. Chem. Int. Ed. Eng. 38:1427-9.
Gryko D T. (2002) Recent advances in the synthesis of corroles and core-modified corroles Eur J Org Chem 11, 1735-1743.
Gryko D. T. and Piechota K. E.(2002) Straightforward route to trans-A2B-corroles bearing substituents with basic nitrogen atoms. J. Porphyrins Phthalocyanines, 6:81.
Gryko D T, Fox J P & Goldberg D P (2004). Recent advances in the chemistry of corroles and core-modified corroles. J. Porph. Phthal. 8:1091-1105.
Haber A., Agadjanian h, Medina-Kauwe L K, and Zeev Gross (2007). Corroles Bind with High Affinity to both Apo and Holo Transferrin. J Inorg Biochem, 102:446.
Haber A, Mahammed A, Fuhrman B, Volkova N, Coleman R, Hayek T, Aviram M and Zeev Gross (2007). Amphiphilic corrole metal complexes as multifunctional agents for attenuation of atherosclerosis. Angew. Chem. Int. Ed. 47:7896.
Han J, Cheng F C, Yang Z, Dryhurst G (1999). Inhibitors of mitochondrial respiration, iron (II), and hydroxyl radical evoke release and extracellular hydrolysis of glutathione in rat striatum and substantia nigra: potential implications to Parkinson's disease. J. Neurochem. 73(4): 1683-95.
Liang, L. P., Huang, J., Fulton, R., Day, B. J. & Patel, M (2007). An orally active catalytic metalloporphyrin protects against 1-methyl-4-phenyl-1,2,3,6-tetrahydropyridine neurotoxicity in vivo. J. Neurosci. 27, 4326-4333.
Mahammed A, Goldberg I & Gross, Z (2001). Highly selective chloro-sulfonation of tris(pentafluorophenyl)corrole as a synthetic tool for the preparation of amphiphilic corroles and metal complexes of chiral planarity, Org. Lett. 3: 3443-3446.
Mahammed A, Gray H B, Weaver J J, Sorasaenee K, and Gross Z. (2004). Amphiphilic Corroles Bind Tightly to Human Serum Albumin” Bioconj Chem. 15:738-746.
Mahammed, A. and Gross, Z. (2005) Albumin-conjugated corrole metal complexes: extremely simple yet very efficient biomimetic oxidation systems. J Am Chem Soc 127, 2883-7.
Mahammed, A. & Gross, Z (2006). Iron and manganese corroles are potent catalysts for the decomposition of peroxynitrite. Angew. Chem. In. Ed Engl 45:6544-6547.
Mandel S, Weinreb O, Youdim M B H (2003). Using cDNA microarray to assess Parkinson's disease models and the effects of neuroprotective drugs. Trends Pharmacol Sci.; 24(4): 184-91.
Mandel S, Amit T, Kalfon L, Youdim MB (2007a). Applying transcriptomic and proteomic knowledge to Parkinson's disease drug discovery. Expert opinion drug discov. 2:1225-40.
Mandel S, Amit T, Bar-Am O, Youdim MB (2007b). Iron disregulation in Alzheimer's disease: multimodal brain permeable iron chelating drugs, possessing neuroprotective-neurorescue and amyloid precursor protein-processing regulatory activities as therapeutic agents. Prog Neurobiol. 82:348-60.
Mohiuddin I, Chai H, Lin P H, Lumsden A B, Yao Q Z, Chen C Y (2006). Nitrotyrosine and chlorotyrosine: Clinical significance and biological functions in the vascular system J Surg Res. 133:143-149.
Motsenbocker M, Ichimori Y and Kondo K (1999). Metal Porphyrin Chemiluminescence Reaction and Application to Immunoassay. Anal. Chem. 65:397-402.
Olmos A, Giner R M, Manez S (2007). Drugs modulating the biological effects of peroxynitrite and related nitrogen species. Med. Res. Rev. 27:1-64.
Peng J., Peng L., Stevenson F. F., Doctrow S. R., and. Andersen J. K (2007). Iron and Paraquat as Synergistic Environmental Risk Factors in Sporadic Parkinson's Disease Accelerate Age-Related Neurodegeneration. J. Neurosc., 27: 6914-6922.
Rha S Y, Izbicka E, Lawrence R, Davidson K, Sun D, Moyer M P, Roodman G D, Hurley L, Von Hoff D. (2000). Effect of telomere and telomerase interactive agents on human tumor and normal cell lines. Clin. Cancer. Res. 6:987-993.
Rohrer L, Hersberger M, von Eckardstein A (2004). High density lipoproteins in the intersection of diabetes mellitus, inflammation and cardiovascular disease. Curr. Opinion Lipidology 15 (3): 269-278.
Sagi, Y. Mandel, S. Amit, T. and Youdim (2007). Activation of tyrosine kinase receptor signaling pathway by rasagiline facilitates neurorescue and restoration of nigrostriatal dopamine neurons in post-MPTP-induced parkinsonism. Neurobiol Dis. 25:35-44.
Saltsman I, Goldberg I & Gross Z (2002) One-step conversions of a simple corrole into chiral and amphiphilic derivatives”, Tetrahedron Lett. 44, 5669-5673.
Saltsman I, Mahammed A, Goldberg I, Tkachenko E, Botoshansky M, Gross Z (2003). Selective substitution of corroles: Nitration, hydroformylation, and chlorosulfonation. J. Am. Chem. Soc. 124:7411-7420.
Saltsman I, Botoshansky M. and Gross Z. (2008) Facile synthesis of ortho-pyridyl-substituted corroles and molecular structures of analogous porphyrins. Tet. Lett, 49:4163
Schlieve, C. R., Lieven, C. J. & Levin, L. A (2006). Biochemical activity of reactive oxygen species scavengers do not predict retinal ganglion cell survival. Invest. Ophthalmol. Vis. Sci. 47:3878-3886.
Shah S. Iqbal M, Karam J, Salifu M, McFarlane S I (2007). Oxidative stress, glucose metabolism, and prevention of type 2 diabetes: pathophysiological insights. Antioxid. Redox Signal. 9:911-929 and references therein.
Szabó C, Ischiropoulos H & Radi R (2007). Peroxynitrite: biochemistry, pathophysiology and development of therapeutics. Nat. Rev. Drug Discov. 6:662-680.
Van der Schyf, C. J., Geldenhuys, W. J. and Youdim, M. B. (2006) Multifunctional drugs with different CNS targets for neuropsychiatric disorders. J. Neurochem. 99, 1033-48.
Weinreb O, Bar-Am O, Amit T, Chillag-Talmor O and Youdim M B H. (2004) Neuroprotection via pro-survival protein kinase C isoforms associated with Bcl-2 family members. FASEB J. 18:1471-3.
Weinstock M, Gorodetsky E, Wang R H, Gross A, Weinreb O, Youdim M B (2002) Limited potentiation of blood pressure response to oral tyramine by brain-selective monoamine oxidase A-B inhibitor, TV-3326 in conscious rabbits. Neuropharmacology 43(6): 999-1005.
Wu A S, Kiaei M, Aguirre N, Crow J P, Calingasan N Y, Browne S E, Beal M F (2003). Iron porphyrin treatment extends survival in a transgenic animal model of amyotrophic lateral sclerosis. J Neurochem 85: 142-150.
Yogev-Falach M, Amit T, Bar-AM O, Youdim M B H (2003). The importance of propargylamine moiety in the anti-Parkinson drug rasagiline and its derivatives for MAPK-dependent amyloid precursor protein processing. FASEB J. 17:(15): 2325-7.
Youdim M B H, Grunblatt E, Mandel S (1999). The pivotal role of iron in NF-kappa B activation and nigrostriatal dopaminergic neurodegeneration. Prospects for neuroprotection in Parkinson's disease with iron chelators. Ann N Y Acad. Sci.; 890:7-25.
Youdim M B (2003). Rasagiline: an anti-Parkinson drug with neuroprotective activity. Expert Rev Neurotherapeutics; 3:(6): 737-749.
Youdim M B, Weinstock M (2004). Therapeutic applications of selective and non-selective inhibitors of monoamine oxidase A and B that do not cause significant tyramine potentiation. Neurotoxicology 25, 243-50.
Youdim M B, Buccafusco J J (2005). Multi-functional drugs for various CNS targets in the treatment of neurodegenerative disorders. Trends Pharmacol. Sci.; 26: 27-35.

1. A method for treatment of diabetes, comprising administering to an individual in need, a transition metal complex of the amphiphilic corrole of the formula I: 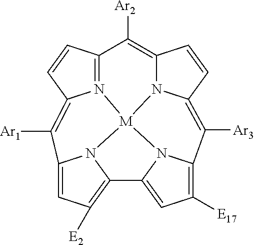
wherein:
Ar₁, Ar₂ and Ar₃, the same or different, each is selected from the group consisting of CF₃, carboaryl, heteroaryl, and mixed carboaryl-heteroaryl;
M is a transition metal selected from the group consisting of Mn, Fe, Ru, Co, V, Cr, and Cu; and
E₂ and E₁₇, the same or different, each is H, SO₃H, SO₂N—R₁R₂, CO₂H, CO₂R or CON—R₁R₂; R is C₁-C₈ alkyl or aryl; and R₁ and R₂, the same or different, each is H, C₁-C₈ alkyl optionally substituted by —COOH, C₂-C₈ alkynyl, C₆-C₁₂ aryl or together with the N atom to which they are attached form a saturated 5-6 membered ring optionally containing a further heteroatom selected from the group consisting of O, S and N, or an optical isomer or a pharmaceutically acceptable salt thereof.

2. The method according to claim 1, wherein said amphiphilic corrole is a 5,10,15-tris-carboaryl-, 5,10,15-tris-heteroaryl or 5,10,15-tris-CF₃-corrole.

3. The method according to claim 1, wherein the carboaryl, by itself or as part of the mixed carboaryl-heteroaryl radical, is a monocyclic or bicyclic C₆-C₁₂ aromatic radical optionally mono- or poly-substituted by one or more halogen atoms, or by C₁-C₈ alkyl or alkoxy, nitro, hydroxyl, SO₃H, —NR₁R₂, —N⁺R₁R₂R₃, or —N—R₁—NH₂, wherein R₁, R₂ and R₃, the same or different, each is H, C₁-C₈ alkyl, C₂-C₈ alkynyl, C₆-C₁₂ aryl, C₆-C₁₂aryl-C₁-C₈ alkyl or R₁ and R₂ together with the N atom to which they are attached form a saturated 5-6 membered ring optionally containing a further heteroatom selected from the group consisting of O, S and N; said heteroaryl, by itself or as part of the mixed carboaryl-heteroaryl radical, is a 5-6 membered aromatic ring containing 1-3 heteroatoms selected from the group consisting of O, S and N optionally substituted as defined above for the carboaryl, and when the heteroaryl has a N atom in the ring, it may be substituted at the N atom, by an alkyl or alkynyl group; and said mixed carboaryl-heteroaryl is a radical derived from a carboaryl and a heteroaryl radical condensed to each other, or covalently linked to each other, optionally substituted as defined above I either in the carbocyclic, heterocyclic, or in both rings.

4. The method according to claim 3, wherein the carboaryl is a phenyl radical monosubstituted by propargylamino or methoxy, or it is polysubstituted, by halogen atoms, sulfo, propargylamino, alkoxy, aminoalkylamino, and trialkylammonium, and the heteroaryl is pyridine substituted at the N atom by C₁-C₄ alkyl, or propargyl.

5. The method according to claim 4, wherein the carboaryl is 2,6-dichlorophenyl, 2,6-difluorophenyl, pentafluorophenyl, 4-methoxy-2,3,5,6-tetrafluorophenyl, 4-sulfophenyl, 4-methoxyphenyl, 4-N-propargylamino-2,3,5,6-tetrafluorophenyl, or 4-N-propargylamino-phenyl; the heteroaryl is 4-(N-methyl)-pyridylium, 2-(N-methyl)-pyridylium, 4-(N-propargyl)-pyridylium, or 2-(N-propargyl)-pyridylium; and the carboaryl-heteroaryl is 4-(pyridyl)-2,3,5,6-tetrafluorophenyl, 4-(N-methyl-pyridylium)-2,3,5,6-tetrafluorophenyl, 4-(N-propargyl-pyridylium)-2,3,5,6-tetra-fluorophenyl, 2-(N-propargyl-pyridylium)-2,3,5,6-tetrafluorophenyl.

6. The method according to claim 5, wherein Ar₁, Ar₂ and Ar₃ are the same and each is CF₃, pentafluorophenyl, 4-methoxy-2,3,5,6-tetrafluorophenyl, 4-sulpho-phenyl, 4-(N-methyl)-pyridylium, 2-(N-methyl)-pyridylium, 4-(N-propargyl)-pyridylium, or 2-(N-propargyl)-pyridylium.

7. The method according to claim 5, wherein Ar₁ and Ar₃ each is 4-(N-methyl)-pyridylium and Ar₂ is pentafluorophenyl; or Ar₁ and Ar₃ each is 4-N-propargylamino-2,3,5,6-tetrafluorophenyl and Ar₂ is 4-methoxyphenyl; or Ar₁ and Ar₃ each is 4-(N-propargyl)-pyridylium and Ar₂ is pentafluorophenyl; or Ar₁ and Ar₃ each is 2-(N-propargyl)-pyridylium and Ar₂ is pentafluorophenyl; or Ar₁ and Ar₃ each is 2-(N-methyl)-pyridylium and Ar₂ is 4-N-propargylaminophenyl.

8. The method according to claim 5, wherein E₂ and E₁₇ are the same and each is H, SO₃H, SO₂NH-propargyl or SO₂NH—CH₂—COOH.

9. The method according to claim 1, wherein the metal M is Fe or Mn.

10. The method according to claim 1, wherein said transition metal complex of the amphiphilic corrole is selected from the group consisting of:
(i) the corroles in which E₂ and E₁₇ are both SO₃H, Ar₁, Ar₂ and Ar₃ each is pentafluorophenyl, and M is Fe (herein designated corrole 1C-Fe), Mn (herein designated corrole 1C-Mn), or Cu (herein designated corrole 1C-Cu); or Ar₁, Ar₂ and Ar₃ each is CF₃ and M is Fe or Mn;
(ii) the corrole in which E₂ and E₁₇ are both SO₃H, Ar₁, Ar₂ and Ar₃ each is 4-methoxy-2,3,5,6,-tetrafluorophenyl, and M is Fe (herein designated corrole 6C-Fe);
(iii) the corrole in which E₂ and E₁₇ are both SO₂NH—CH₂—COOH, Ar₁, Ar₂ and Ar₃ each is pentafluorophenyl, and M is Fe (herein designated corrole 5C-Fe);
(iv) the corrole in which E₂ and E₁₇ are both H, Ar₁ and Ar₃ each is 4-(N-methyl)-pyridylium, Ar₂ is pentafluorophenyl, and M is Mn (herein designated corrole 2C-Mn);
(v) the corrole in which E₂ and E₁₇ are both H, Ar₁, Ar₂ and Ar₃ each is 2-(N-methyl)-pyridylium, and M is Mn (herein designated corrole 3C-Mn);
(vi) the corrole in which E₂ and E₁₇ are both H, Ar₁ and Ar₃ each is 2-(N-methyl)-pyridylium, Ar₂ is pentafluorophenyl, and M is Mn (herein designated corrole 4C-Mn); and
(vii) the corroles in which E₂ and E₁₇ are both SO₃H, Ar₁, Ar₂ and Ar₃ each is CF₃, and M is Mn or Fe (herein designated corrole M-Mn or H-Fe, respectively);
(viii) the corroles in which E₂ and E₁₇ are both H, Ar₁ and Ar₃ each is 4-(N-propargyl)-pyridylium, Ar₂ is pentafluorophenyl and M is Mn or Fe (herein designated corrole E-pr-Mn or E-pr-Fe, respectively);
(ix) the corrole in which E₂ and E₁₇ are both H, Ar₁ and Ar₃ each is 2-(N-propargyl)-pyridylium, Ar₂ is pentafluorophenyl, and M is Mn or Fe (herein designated corrole H-Mn or H-Fe, respectively);
(x) the corroles in which E₂ and E₁₇ are both SO₃H, Ar₁ and Ar₃ each is 4-N-propargylamino-2,3,5,6-tetrafluorophenyl and Ar₂ is 4-methoxyphenyl, and M is Mn or Fe (herein designated corrole I-Mn or I-Fe, respectively);
(xi) the corrole in which E₂ and E₁₇ are both H, Ar₁ and Ar₃ each is 2-(N-methyl)-pyridylium, Ar₂ is 4-propargylamino-phenyl, and M is Mn or Fe (herein designated corrole J-Mn or J-Fe, respectively);
(xii) the corroles in which E₂ and E₁₇ are both —SO₂—NH-propargyl, Ar₁, Ar₂ and Ar₃ each is pentafluorophenyl, and M is Mn or Fe (herein designated corrole K-Mn or K-Fe, respectively); and
(xiii) the corroles in which E₂ and E₁₇ are both —SO₂—NH-propargyl, Ar₁, Ar₂ and Ar₃ each is CF₃, and M is Mn or Fe (herein designated corrole M-Mn or M-Fe, respectively).

11. The method according to claim 10, wherein said corrole is selected from the group consisting of the corroles designated 1C-Fe, 1C-Mn, 2C-Mn, 3C-Mn, 4C-Mn, 5C-Fe, 5C-Fe, and E-pr-Mn.

12. The method according to claim 1 wherein administering transition metal complex of the amphiphilic corrole of the formula I inhibits cellular death or cellular damage in pancreatic beta cells.
